(19) United States
(12) Patent Application Publication
(10) Pub. No.: US 2011/0015280 A1
(43) Pub. Date: Jan. 20, 2011

(54) AMPHOLYTIC COPOLYMER, PRODUCTION THEREOF, AND USE OF THE SAME

(75) Inventors: Son Nguyen Kim, Hemsbach (DE); Ivette Garcia Castro, Ludwigshafen (DE); Klemens Mathauer, Heidelberg (DE)

Correspondence Address: CONNOLLY BOVE LODGE & HUTZ LLP, 1875 EYE STREET, N.W., SUITE 1100, WASHINGTON, DC, 20006, US

(73) Assignee: BASF AKTIENGESELLSCHAFT, Ludwigshafen (DE)

(21) Appl. No.: 11/996,436

(22) PCT Filed: Jul. 21, 2006
(86) PCT No.: PCT/EP2006/064504

§ 371 Date: Oct. 6, 2010

(30) Foreign Application Priority Data
Jul. 22, 2005 (DE) 10 2005 034 412.7

(51) Int. Cl. A61K 47/32; C08F 226/06

(52) U.S. Cl. 514/772.5; 526/258

(57) ABSTRACT

The present invention relates to an ampholytic copolymer, to a process for its preparation, to cosmetic or pharmaceutical compositions which comprise at least one such ampholytic copolymer, and to further uses of these copolymers.

The present invention relates to an ampholytic copolymer, to a process for its preparation, to cosmetic or pharmaceutical compositions which comprise at least one such ampholytic copolymer, and to further uses of these copolymers.

Polymers with a relatively large number of ionically dissociatable groups in the main chain and/or a side chain are referred to as polyelectrolytes. If these polymers have both anionogenic/anionic groups and also cationogenic/cationic groups, then these are amphoteric polyelectrolytes or ampholytic polymers. Ampholytic polymers with a sufficient number of dissociatable groups are water-soluble or water-dispersible and have found diverse applications in the field of coatings, paper auxiliaries, hygiene compositions, in the manufacture of textiles, and especially in pharmacy and cosmetics.

Cosmetically and pharmaceutically acceptable water-soluble polymers serve, for example in soaps, creams and lotions, as formulating agents, e.g. as thickener, foam stabilizer or water absorbent, or else to alleviate the irritative effect of other ingredients or to improve the dermal application of active ingredients. Their task in hair cosmetics consists in influencing the properties of the hair. In pharmacy, they serve, for example, as coatings or binders for solid medicaments. For hair cosmetics, film-forming polymers with ionic groups are used, for example, as conditioners for improving the dry and wet combability, the feel to the touch, the shine and the appearance of the hair, and also for imparting antistatic properties to the hair. Depending on the intended application, water-soluble polymers with cationic or anionic functionalities are used here. Thus, polymers with cationic functional groups have a high affinity to the negatively charged surface of the hair as a result of their structure. Polymers with anionic functionalities, such as, for example, if appropriate crosslinked polyacrylic acid, serve, for example, as thickeners, in addition polymers containing carboxylate groups are used, for example, for setting hairstyles.

The provision of products with a complex profile of properties often presents difficulties. For example, there is a need for polymers for cosmetic and other compositions which have good film-forming properties and at the same time allow the rheological properties of the compositions to be adjusted. In addition, esthetic requirements are increasingly being made on cosmetic and pharmaceutical products by the consumer. For example, with products of this type, a preference for clear, opaque formulations in the form of gels is currently observed. In many cases, the desired profile of properties can only be achieved by using a plurality of polymers with ionic groups. However, the various polymers are often incompatible with one another, which can, for example, lead to undesired salting out. There is therefore a need for cosmetically and pharmaceutically compatible ampholytic polymers which are suitable, when used as the sole polymer component, for providing a certain profile of properties with regard to film formation and rheology and/or which are highly compatible with a large number of different polyelectrolytes.

Furthermore, active substances for cosmetics, but also for medicaments, hygiene products, material protection etc., i.e. substances which develop an effect even at low concentration, e.g. a cosmetic effect on the skin or the hair, pharmacological effect within an organism etc., are often formulated and used in the form of aqueous active substance preparations. Alternatively, formulation and administration is also possible in solid form, e.g. as powder or compact (tablet, etc.), in which case, however, transportation to the actual site of action involves conversion to an aqueous form. A principal problem with aqueous active substance preparations is the low solubility in water of many active ingredients, which is often less than 5 g/l at 23° C./1013 mbar. Here too, there is a continual need for cosmetically and pharmaceutically compatible ampholytic polymers which serve at the same time as solubilizers for components which are insoluble or only slightly soluble in water.

U.S. Pat. No. 3,915,921 describes copolymers which comprise, in copolymerized form, an olefinically unsaturated carboxylic acid, a C₁₀-C₃₀-alkyl (meth)acrylate and, if appropriate, a crosslinking monomer with at least two ethylenically unsaturated double bonds. In neutralized form, they serve as thickeners for diverse applications.

WO 97/21744 describes crosslinked anionic copolymers and their use as thickeners and dispersants in aqueous systems.

EP-A-0 982 021 describes the use of (partially) neutralized copolymers of
A) 50 to 99% by weight of monoethylenically unsaturated carboxylic acids and
B) 1 to 50% by weight of at least one comonomer chosen from
   a) monoethylenically unsaturated carboxylic esters with saturated C₈-C₃₀-alcohols,
   b) N-C₈-C₁₈-alkyl- and N,N-di-C₈-C₁₈-alkylcarboxamides,
   c) vinyl esters of aliphatic C₈-C₃₀-carboxylic acids,
   d) C₈-C₁₈-alkyl vinyl ethers,
   and mixtures thereof

as thickeners for producing hair-washing compositions.

WO 01/62809 describes a cosmetic composition which comprises at least one water-soluble or water-dispersible polymer which comprises, in incorporated form,
a) 5 to 50% by weight of at least one α,β-ethylenically unsaturated monomer with a tert-butyl group,
b) 25 to 90% by weight of at least one N-vinylamide and/or N-vinyllactam,
c) 0.5 to 30% by weight of at least one compound with a free-radically polymerizable, α,β-ethylenically unsaturated double bond and at least one cationogenic and/or cationic group per molecule, and
d) 0 to 30% by weight of at least one further α,β-ethylenically unsaturated compound, which may be compounds with at least one anionogenic and/or anionic group per molecule.

EP-A-670 333 describes crosslinked water-soluble polymer dispersions which are obtainable by polymerization of a monomer mixture comprising at least one water-soluble monomer, at least one crosslinker, and, if appropriate, hydrophobic and/or amphiphilic monomers in the presence of a polymeric dispersant. Besides a large number of others, water-soluble monomers which can be used are also N-vinyl-pyrrolidone, and monomers with cationic/cationizable groups, such as N-vinylimidazole. The polymeric dispersants may be polyelectrolytes which comprise, in copolymerized form, for example, salts of (meth)acrylic acid as anionic monomer building blocks or quaternized derivatives of N,N-dimethylaminoethyl(meth)acrylate as cationic building blocks. Use of the polymer dispersions in cosmetics is not described.

EP-A-929 285 teaches the use of water-soluble copolymers which comprise, in copolymerized form, vinylcarboxamide units and vinylimidazole units as a constituent of cosmetic compositions. Polyelectrolyte complexes of these copolymers with polymers containing carboxylic acid groups are not disclosed in this document.

WO 00/27893 describes aqueous polymer dispersions based on N-vinylcarboxamides and, if appropriate, comonomers, in which case, besides a large number of others, mention is also made of N-vinylpyrrolidone, N-vinylimidazole and N-vinylimidazole derivatives. The polymerization takes place in the presence of at least one polymeric dispersant. Use of these polymer dispersions in cosmetics is described only very generally and without being demonstrated by a working example.

EP-A-1038891 describes water-soluble or water-dispersible polymeric salts of at least one polymer and at least one oppositely charged neutralizing agent.

WO 02/41856 describes the use of polymer dispersions which are obtainable by polymerization of at least one water-soluble monomer in an aqueous salt solution which comprises at least one polyelectrolyte as dispersant for the cosmetic treatment of keratin materials. In addition, the dispersions comprise at least one agent for adjusting the viscosity, for example a polycarboxylic acid or a salt thereof. Water-soluble monomers which can be used are cationic, anionic and nonionic monomers, preference being given to monomer mixtures which comprise at least one cationic monomer and, if appropriate, in addition acrylic acid and/or acrylamide.

WO 2005/004821 describes a cosmetic or pharmaceutical composition which comprises at least one polyelectrolyte complex which comprises at least one water-soluble or water-dispersible copolymer A1) with cationogenic groups, which comprises, in copolymerized form, vinylimidazole and/or a derivative thereof and at least one further monomer copolymerizable therewith, and at least one polymer A2) containing acid groups.

WO 2005/005497 describes an aqueous polymer dispersion Pd) which is obtainable by free-radical polymerization of a monomer mixture M) comprising
a) at least one α,β-ethylenically unsaturated amide-group-containing compound of the general formula I

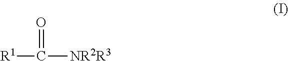

where
R² is a group of the formula CH₂═CR⁴— and R¹ and R³, independently of one another, are H, alkyl, cycloalkyl, heterocycloalkyl, aryl or hetaryl, or R¹ and R³ together with the amide group to which they are bonded are a lactam having 5 to 8 ring atoms,
b) at least one free-radically polymerizable crosslinking compound with at least two α,β-ethylenically unsaturated double bonds per molecule,
c) at least one compound with a free-radically polymerizable α,β-ethylenically unsaturated double bond and at least one cationogenic and/or cationic group per molecule,

in an aqueous medium in the presence of at least one polymeric anionic dispersant D). Also described are cosmetic or pharmaceutical compositions which comprise such a polymer dispersion or a polymer obtainable by drying such a dispersion.

WO 00/39176 describes a hydrophilic, cationic, ampholytic copolymer which comprises, in copolymerized form, 0.05 to 20 mol % of an anionic monomer with at least one carboxy group, 0 to 45 mol % of a cationic monomer with at least one amino group and, if appropriate, a hydrophobic monomer and/or a crosslinker, where the molar ratio of cationic to anionic monomer is about 2:1 to 16:1. These ampholytic copolymers can be used inter alia for modifying rheological properties of bodycare compositions.

WO 04/058837 describes an ampholytic copolymer which is obtainable by free-radical copolymerization of
a) at least one ethylenically unsaturated compound with at least one anionogenic and/or anionic group,
b) at least one ethylenically unsaturated compound with at least one cationogenic and/or cationic group,
c) at least one unsaturated compound containing amide groups

and, if appropriate, further comonomers. Also described are polyelectrolyte complexes which comprise such an ampholytic copolymer, and cosmetic or pharmaceutical compositions based on these ampholytic copolymers and polyelectrolyte complexes.

US 2006/0084586 A1 describes rheology-modifying hair-setting resins which comprise a crosslinked copolymer based on vinylamide and carboxylic acid monomers. Polymers which comprise, in copolymerized form, at least one cationic compound chosen from N-vinylimidazole compounds, N-[3-(dimethylamino)propyl]acrylamide, N-[3-(dimethylamino)propyl]methacrylamide and mixtures thereof are not described.

There continues to be a need for polymers which, besides good film-forming properties, also allow the rheological properties of the products to be adjusted, meaning that they can be formulated, for example, in the form of mousses or gels. Also desirable are polymers which are suitable as solubilizers for components which are insoluble or only slightly soluble in water and/or are characterized by good compatibility with other formulation constituents. In addition, polymers for use in hair cosmetics should be characterized by further advantageous properties, such as for example the production of elastic hairstyles with simultaneously strong hold even at high atmospheric humidity, good ability to be washed out and by good feel of the hair treated therewith.

The invention firstly provides an ampholytic copolymer A) obtainable by free-radical copolymerization of
a) at least one compound with a free-radically polymerizable, α,β-ethylenically unsaturated double bond and at least one anionogenic and/or anionic group per molecule,
b) at least one compound which is chosen from N-vinylimidazole compounds, N-[3-(dimethylamino)propyl]acrylamide, N-[3-(dimethylamino)propyl]methacrylamide and mixtures thereof,
c) at least one free-radically polymerizable crosslinking compound which comprises at least two α,β-ethylenically unsaturated double bonds per molecule.

In particular at least part of the compounds a) and b) is used in the form of a monomer pair, where the molar ratio of anionogenic groups of component a) to cationogenic groups of component b) is about 1:1.

The ampholytic copolymers A) according to the invention can be prepared by conventional polymerization processes, e.g. by solution polymerization, emulsion polymerization, suspension polymerization or bulk polymerization. Copolymers with particularly advantageous properties, e.g. with generally higher molecular weights and a better capability as rheology modifiers (specifically as thickeners) than are achieved by conventional polymerization processes are achieved by the preparation in accordance with the method of precipitation polymerization. A preferred embodiment of the invention are therefore ampholytic copolymers A) which are obtainable by free-radical copolymerization in accordance with the method of precipitation polymerization. In a specific embodiment, for the production of the copolymers according to the invention, use is made of at least two free-radical initiators whose decomposition temperatures and/or whose half-lives at a certain polymerization temperature are different from one another. In this case, copolymers with particularly small residual monomer contents can be achieved. This is the case particularly if the initiator which decomposes at the higher temperature is added before completion, preferably before the start of the precipitation of the polymer.

In the precipitation polymerization, the monomers used are soluble in the reaction medium (monomer, solvent), but the corresponding polymer is not. The polymer which forms becomes unsoluble under the chosen polymerization conditions and precipitates out of the reaction mixture.

For the purposes of the present invention, the expression alkyl comprises straight-chain and branched alkyl groups. Suitable short-chain alkyl groups are, for example, straight-chain or branched C₁-C₇-alkyl groups, preferably C₁-C₆-alkyl groups and particularly preferably C₁-C₄-alkyl groups. These include, in particular, methyl, ethyl, propyl, isopropyl, n-butyl, 2-butyl, sec-butyl, tert-butyl, n-pentyl, 1-methylbutyl, 2-methylbutyl, 3-methylbutyl, 1,2-dimethylpropyl, 1,1-dimethylpropyl, 2,2-dimethyl-propyl, 1-ethylpropyl, n-hexyl, 1-methylpentyl, 2-methylpentyl, 3-methylpentyl, 4-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, n-heptyl, 2-heptyl, 3-heptyl, 2-ethylpentyl, 1-propylbutyl, octyl etc.

Suitable longer-chain C₈-C₃₀-alkyl groups and C₈-C₃₀-alkenyl groups are straight-chain and branched alkyl and alkenyl groups. Preference is given here to predominantly linear alkyl radicals, as also occur in natural or synthetic fatty acids and fatty alcohols and in oxo alcohols, which may, if appropriate, additionally be mono-, di- or polyunsaturated. These include, for example, n-hexyl(ene), n-heptyl(ene), n-octyl(ene), n-nonyl(ene), n-decyl(ene), n-undecyl(ene), n-dodecyl(ene), n-tridecyl(ene), n-tetradecyl(ene), n-pentadecyl(ene), n-hexadecyl(ene), n-heptadecyl(ene), n-octadecyl(ene), n-nonadecyl(ene), arachinyl(ene), behenyl(ene), lignocerinyl(ene), melissinyl(ene), etc.

Cycloalkyl is preferably C₅-C₈-cycloalkyl, such as cyclopentyl, cyclohexyl, cycloheptyl or cyclooctyl.

Aryl comprises unsubstituted and substituted aryl groups and is preferably phenyl, tolyl, xylyl, mesityl, naphthyl, fluorenyl, anthracenyl, phenanthrenyl, naphthacenyl and in particular phenyl, tolyl, xylyl or mesityl.

In the text below, compounds which are derived from acrylic acid and methacrylic acid may sometimes be referred to in short by adding the syllable “(meth)” to the compound derived from acrylic acid.

The copolymers A) according to the invention can advantageously be formulated as gels under normal conditions (20° C.). “Gel-like consistency” is shown by formulations which have a higher viscosity than a liquid and which are self-supporting, i.e. which retain a shape imparted to them without a shape-stabilizing covering. In contrast to solid formulations, gel-like formulations can, however, easily be deformed under the application of shear forces. The viscosity of the gel-like compositions is preferably in a range from greater than 600 to about 60 000 mPas, particularly preferably from 6000 to 30 000 mPas. The gels are preferably hair gels.

For the purposes of the present invention, water-soluble monomers and polymers are understood as meaning monomers and polymers which dissolve in water at 20° C. in an amount of at least 1 g/l. Water-dispersible monomers and polymers are understood as meaning monomers and polymers which disintegrate into dispersible particles under the application of shear forces, for example by stirring. Hydrophilic monomers are preferably water-soluble or at least water-dispersible. The copolymers A) according to the invention are generally water-soluble.

The ampholytic copolymers A) according to the invention have both anionogenic and/or anionic groups, and also cationogenic and/or cationic groups. To prepare them, preferably part of the monomers a) and b) is used together, i.e. in the form of a monomer pair (“monomer salt”). In this monomer composition, the molar ratio of anionogenic and anionic groups of component a) to cationogenic and cationic groups of component b) is about 1:1 (i.e. monovalent monomers are essentially used in equimolar amounts). The monomer pairs can be prepared separately prior to being used for the polymerization. However, preference is given to the “in situ” preparation of the monomer pairs through joint use (e.g. joint feed) during the preparation of the copolymers.

Preferably, the amount of compounds used for the polymerization in the monomer pair is at least 1% by weight, preferably at least 2% by weight, in particular at least 3% by weight.

Further monomers with ionogenic/ionic groups which are not used in the form of the monomer pair for the polymerization are preferably used in initially uncharged form, i.e. with anionogenic or cationogenic groups. If desired, for the preparation of the ampholytic copolymers according to the invention it is possible to also use already charged monomers, i.e. monomers with anionic and cationic groups, instead of uncharged monomers or in addition to uncharged monomers or in addition to the monomer pairs. The counterions which these monomers carry are then preferably derived from acids or bases, as are described below for adjusting the pH during the polymerization or of the polymers obtained. Cationic monomers are, in addition, used in partially or completely quaternized form.

Preferably, the copolymers A) have a clear excess of anionogenic/anionic groups or of cationogenic/cationic groups. In a specific embodiment, for the copolymerization, monomers with ionogenic or ionic groups are used in amounts such that the copolymer A) has a molar excess of anionogenic/anionic groups compared with cationogenic/cationic groups or a molar excess of cationogenic/cationic groups compared with anionogenic/anionic groups of at least 1.2:1, particularly preferably at least 1.4:1, in particular at least 2:1, especially at least 2.5:1.

In particular, the copolymers A) according to the invention comprise no silicon-atom-containing groups in incorporated form.

Monomer a)

The copolymers A) according to the invention comprise, in copolymerized form, as compound a), at least one compound with a free-radically polymerizable α,β-ethylenically unsaturated double bond and at least one anionogenic and/or anionic group per molecule. The component a) can be used in an amount of from 1 to 96% by weight, such as from 2 to 90% by weight, based on the total weight of the compounds used for the polymerization. The component a) is preferably used in an amount of from 2 to 70% by weight, preferably 3 to 60% by weight, based on the total weight of the compounds used for the polymerization (i.e. components a), b), c) and, if present, d) to h)).

Preferably, the compounds a) are chosen from monoethylenically unsaturated carboxylic acids, sulfonic acids, phosphonic acids and mixtures thereof.

The monomers a) include monoethylenically unsaturated mono- and dicarboxylic acids having 3 to 25, preferably 3 to 6, carbon atoms, which can also be used in the form of their salts or anhydrides. Examples thereof are acrylic acid, methacrylic acid, ethacrylic acid, α-chloroacrylic acid, crotonic acid, maleic acid, maleic anhydride, itaconic acid, citraconic acid, mesaconic acid, glutaconic acid, aconitic acid and fumaric acid. The monomers a) also include the half-esters of monoethylenically unsaturated dicarboxylic acids having 4 to 10, preferably 4 to 6, carbon atoms, e.g. of maleic acid, such as monomethyl maleate. The monomers a) also include monoethylenically unsaturated sulfonic acids and phosphonic acids, for example vinylsulfonic acid, allylsulfonic acid, sulfoethyl acrylate, sulfoethyl methacrylate, sulfopropyl acrylate, sulfopropyl methacrylate, 2-hydroxy-3-acryloxypropylsulfonic acid, 2-hydroxy-3-methacryloxy-propylsulfonic acid, styrenesulfonic acid, 2-acrylamido-2-methylpropanesulfonic acid, vinylphosphonic acid and allylphosphonic acid. The monomers a) also include the salts of the abovementioned acids, in particular the sodium, potassium and ammonium salts, and also the salts with amines. The monomers a) can be used as they are or as mixtures with one another. The weight fractions given all refer to the acid form.

Preferably, the component a) is chosen from acrylic acid, methacrylic acid, ethacrylic acid, α-chloroacrylic acid, crotonic acid, maleic acid, maleic anhydride, fumaric acid, itaconic acid, citraconic acid, mesaconic acid, glutaconic acid, aconitic acid and mixtures thereof.

The component a) is particularly preferably chosen from acrylic acid, methacrylic acid and mixtures thereof.

Monomer b)

The copolymers according to the invention comprise preferably 2 to 96% by weight, particularly preferably 2 to 90% by weight, especially preferably 3 to 70% by weight, based on the total weight of the monomers used for the polymerization, of at least one monomer b) in copolymerized form. Suitable monomers b) are compounds of the formula

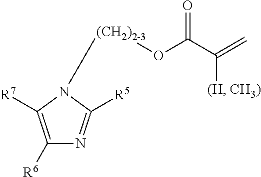

in which R⁵ to R⁷, independently of one another, are hydrogen, C₁-C₄-alkyl or phenyl. Preferably, R⁵ to R⁷ are hydrogen.

Furthermore, the copolymer preferably comprises, as monomer b), at least one N-vinylimidazole compound of the general formula (II)

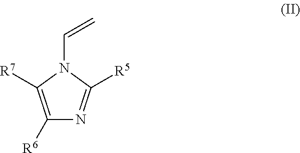

in copolymerized form, in which R⁵ to R⁷, independently of one another, are hydrogen, C₁-C₄-alkyl or phenyl.

Examples of compounds of the general formula (II) are given in table 1 below:

TABLE 1

| R⁵ | R⁶ | R⁷ |
|---|---|---|
| H | H | H |
| Me | H | H |
| H | Me | H |
| H | H | Me |
| Me | Me | H |
| H | Me | Me |
| Me | H | Me |
| Ph | H | H |
| H | Ph | H |
| H | H | Ph |
| Ph | Me | H |
| Ph | H | Me |
| Me | Ph | H |
| H | Ph | Me |
| H | Me | Ph |
| Me | H | Ph |

Me = methyl
Ph = phenyl

As monomer b), preference is given to 1-vinylimidazole (N-vinylimidazole).

Suitable monomers b) are also the compounds obtainable by protonation or quaternization of the abovementioned N-vinylimidazole compounds. Examples of such charged monomers b) are quaternized vinylimidazoles, in particular 3-methyl-1-vinyl-imidazolium chloride and methosulfate. Suitable acids and alkylating agents are listed below for component f).

A specific embodiment covers copolymers A) which comprise, in copolymerized form, at least one N-vinylimidazole compound, specifically N-vinylimidazole, as sole monomer b). If at least one N-vinylimidazole compound, specifically N-vinylimidazole, is used as sole monomer b), then the fraction is preferably 3 to 96% by weight, based on the total weight of the compounds used for the polymerization.

Suitable monomers b) are also N-[3-(dimethylamino)propyl]acrylamide, N-[3-(dimethylamino)propyl]methacrylamide and mixtures thereof.

A preferred monomer b) is N-[3-(dimethylamino)propyl]methacrylamide (DMAPMAM).

A specific embodiment covers copolymers A) which comprise, in copolymerized form, N-[3-(dimethylamino)propyl]acrylamide and/or N-[3-(dimethylamino)propyl]methacryl-amide, specifically DMAPMAM, as sole monomer b). The fraction of N-[3-(dimethylamino)propyl]acrylamide and N-[3-(dimethylamino)propyl]methacrylamide (in total, if both are present) is then preferably 2 to 95% by weight, particularly preferably 3 to 60% by weight, based on the total weight of the monomers used for the polymerization.

Instead of or in addition to the abovementioned compounds b), the copolymers A) can comprise, in copolymerized form, at least one other monomer f) having at least one cationogenic and/or cationic group. The fraction of these monomers f) is preferably 0 to 50% by weight, particularly preferably 0 to 30% by weight, very particularly preferably 0.1 to 20% by weight, based on the total weight of the compounds used for the polymerization.

Crosslinker c)

The copolymers A) can, if desired, comprise at least one crosslinker, i.e. a compound with two or more than two ethylenically unsaturated, nonconjugated double bonds in copolymerized form.

Preferably, crosslinkers are used in an amount of from 0.01 to 5% by weight, particularly preferably 0.1 to 4% by weight, based on the total weight of the monomers used for the polymerization.

Suitable crosslinkers c) are, for example, acrylic esters, methacrylic esters, allyl ethers or vinyl ethers of at least dihydric alcohols. The OH groups of the parent alcohols here may be completely or partially etherified or esterified; however, the crosslinkers comprise at least two ethylenically unsaturated groups.

Examples of the parent alcohols are dihydric alcohols, such as 1,2-ethanediol, 1,2-propanediol, 1,3-propanediol, 1,2-butanediol, 1,3-butanediol, 2,3-butanediol, 1,4-butanediol, but-2-ene-1,4-diol, 1,2-pentanediol, 1,5-pentanediol, 1,2-hexanediol, 1,6-hexanediol, 1,10-decanediol, 1,2-dodecanediol, 1,12-dodecanediol, neopentyl glycol, 3-methylpentane-1,5-diol, 2,5-dimethyl-1,3-hexanediol, 2,2,4-trimethyl-1,3-pentanediol, 1,2-cyclohexanediol, 1,4-cyclohexanediol, 1,4-bis(hydroxymethyl)cyclohexane, hydroxypivalic neopentyl glycol monoester, 2,2-bis(4-hydroxyphenyl)propane, 2,2-bis[4-(2-hydroxypropyl)phenyl]propane, diethylene glycol, triethylene glycol, tetraethylene glycol, dipropylene glycol, tripropylene glycol, tetrapropylene glycol, 3-thiopentane-1,5-diol, and polyethylene glycols, polypropylene glycols and polytetrahydrofurans with molecular weights of in each case 200 to 10 000. Apart from the homopolymers of ethylene oxide and propylene oxide, it is also possible to use block copolymers of ethylene oxide or propylene oxide or copolymers which comprise incorporated ethylene oxide and propylene oxide groups. Examples of parent alcohols with more than two OH groups are trimethylolpropane, glycerol, pentaerythritol, 1,2,5-pentanetriol, 1,2,6-hexanetriol, triethoxycyanuric acid, sorbitan, sugars, such as sucrose, glucose, mannose. The polyhydric alcohols can of course also be used following reaction with ethylene oxide or propylene oxide as the corresponding ethoxylates or propoxylates. The polyhydric alcohols can also firstly be converted to the corresponding glycidyl ethers by reaction with epichlorohydrin. Preference is given to ethylene glycol di(meth)acrylate and polyethylene glycol di(meth)acrylates.

Further suitable crosslinkers c) are the vinyl esters or the esters of monohydric, unsaturated alcohols with ethylenically unsaturated C₃-C₆-carboxylic acids, for example acrylic acid, methacrylic acid, itaconic acid, maleic acid or fumaric acid. Examples of such alcohols are allyl alcohol, 1-buten-3-ol, 5-hexen-1-ol, 1-octen-3-ol, 9-decen-1-ol, dicyclopentenyl alcohol, 10-undecen-1-ol, cinnamyl alcohol, citronellol, crotyl alcohol or cis-9-octadecen-1-ol. However, it is also possible to esterify the monohydric, unsaturated alcohols with polybasic carboxylic acids, for example malonic acid, tartaric acid, trimellitic acid, phthalic acid, terephthalic acid, citric acid or succinic acid.

Further suitable crosslinkers c) are esters of unsaturated carboxylic acids with the above-described polyhydric alcohols, for example of oleic acid, crotonic acid, cinnamic acid or 10-undecenoic acid.

Suitable crosslinkers c) are also straight-chain or branched, linear or cyclic, aliphatic or aromatic hydrocarbons which have at least two double bonds, which, in the case of aliphatic hydrocarbons, must not be conjugated, e.g. divinylbenzene, divinyltoluene, 1,7-octadiene, 1,9-decadiene, 4-vinyl-1-cyclohexene, trivinylcyclohexane or polybutadienes with molecular weights of from 200 to 20 000.

Also suitable as crosslinkers c) are the acrylamides, methacrylamides and N-allylamines of at least difunctional amines. Such amines are, for example, 1,1-diaminomethane, 1,2-diaminoethane, 1,3-diaminopropane, 1,4-diaminobutane, 1,6-diaminohexane, 1,12-dodecanediamine, piperazine, diethylenetriamine or isophoronediamine. Likewise suitable are the amides of allylamine and unsaturated carboxylic acids, such as acrylic acid, methacrylic acid, itaconic acid, maleic acid, or at least dibasic carboxylic acids, as have been described above.

In addition, triallylamine and triallylmonoalkylammonium salts, e.g. triallylmethylammonium chloride or methyl sulfate, are suitable as crosslinkers c).

Also suitable are N-vinyl compounds of urea derivatives, at least difunctional amides, cyanurates or urethanes, for example of urea, ethyleneurea, propyleneurea or tartardiamide, e.g. N,N′-divinylethyleneurea or N,N′-divinylpropyleneurea.

Further suitable crosslinkers c) are divinyldioxane, tetraallylsilane or tetravinylsilane.

It is of course also possible to use mixtures of the abovementioned compounds c).

As crosslinkers c), very particular preference is given to ethylene glycol di(meth)acrylate, polyethylene glycol di(meth)acrylates, pentaerythritol triallyl ether, methylenebisacrylamide, N,N′-divinylethyleneurea, triallylamine and triallylmonoalkylammonium salts.

Monomer d)

In a preferred embodiment, the copolymers according to the invention comprise, in copolymerized form, in addition to the abovementioned monomers a) to c), at least one further monomer d) containing amide groups and of the general formula I

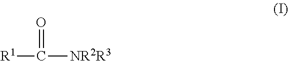

where

one of the radicals R¹ to R³ is a group of the formula CH₂═CR⁴—, where R⁴═H or C₁-C₄-alkyl, and the other radicals R¹ to R³, independently of one another, are H, alkyl, cycloalkyl, heterocycloalkyl, aryl or hetaryl,

where R¹ and R², together with the amide group to which they are bonded, may also be a lactam having 5 to 8 ring atoms,

where R² and R³, together with the nitrogen atom to which they are bonded, may also be a five- to seven-membered heterocycle,

with the proviso that the sum of the carbon atoms of the radicals R¹, R² and R³ is at most 8.

Preferably, in addition to the carbonyl carbon atom of the amide group, the compounds of component d) have at most 7 further carbon atoms.

Preferably, the compounds of component d) are chosen from primary amides of α,β-ethylenically unsaturated monocarboxylic acids, N-vinylamides of saturated monocarboxylic acids, N-vinyllactams, N-alkylamides and N,N-dialkylamides of α,β-ethylenically unsaturated monocarboxylic acids and mixtures thereof.

Preferred mononers d) are N-vinyllactams and derivatives thereof, which can, for example, have one or more C₁-C₆-alkyl substituents, such as methyl, ethyl, n-propyl, isopropyl, n-butyl, sec-butyl, tert-butyl etc. These include, for example, N-vinyl-pyrrolidone, N-vinylpiperidone, N-vinylcaprolactam, N-vinyl-5-methyl-2-pyrrolidone, N-vinyl-5-ethyl-2-pyrrolidone, N-vinyl-6-methyl-2-piperidone, N-vinyl-6-ethyl-2-piperidone, N-vinyl-7-methyl-2-caprolactam, N-vinyl-7-ethyl-2-caprolactam etc.

Particular preference is given to using N-vinylpyrrolidone and/or N-vinylcaprolactam.

Suitable monomers d) are also acrylamide and methacrylamide.

Suitable N-alkylamides and N,N-dialkylamides of α,β-ethylenically unsaturated monocarboxylic acids which, in addition to the carbonyl carbon atom of the amide group, have at most 7 further carbon atoms are, for example, N-methyl(meth)acrylamide, N-ethyl(meth)acrylamide, N-propyl(meth)acrylamide, N-(n-butyl)(meth)acrylamide, N-tert-butyl(meth)acrylamide, n-pentyl(meth)acrylamide, n-hexyl(meth)acrylamide, n-heptyl(meth)acrylamide, N,N-dimethyl(meth)acrylamide, N,N-diethyl(meth)acrylamide, piperidinyl(meth)acrylamide, morpholinyl(meth)acryl-amide and mixtures thereof.

Open-chain N-vinylamide compounds suitable as monomers d) are, for example, N-vinylformamide, N-vinyl-N-methylformamide, N-vinylacetamide, N-vinyl-N-methyl-acetamide, N-vinyl-N-ethylacetamide, N-vinylpropionamide, N-vinyl-N-methyl-propionamide, N-vinyl-butyramide and mixtures thereof. Preference is given to using N-vinylformamide.

Suitable monomers d) are also compounds of the formula

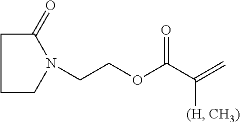

The copolymers A) according to the invention comprise preferably 5 to 95% by weight, particularly preferably 10 to 90% by weight, based on the total weight of the compounds used for the polymerization, of at least one monomer d) in copolymerized form.

Monomer e)

The copolymers A) according to the invention comprise, in copolymerized form, preferably 0.2 to 50% by weight, particularly preferably 0.5 to 40% by weight, in particular 1 to 30% by weight, based on the total weight of the compounds used for the polymerization, of at least one hydrophobic monomer e) (and/or at least one hydrophobic monomer g)).

Suitable compounds e) are chosen from compounds of the general formulae III a), III b), III c), III d) and III e)

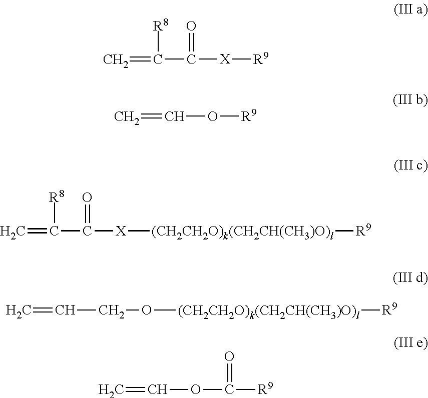

in which
the order of the alkylene oxide units is arbitrary,
k and l, independently of one another, are an integer from 0 to 1000, where the sum of k and l is at least 5,
R⁸ is hydrogen or C₁-C₄-alkyl, preferably methyl,
R⁹ is C₈-C₃₀-alkyl or C₈-C₃₀-alkenyl, and
X is O or a group of the formula NR¹⁰, in which R¹⁰ is H, alkyl, alkenyl, cycloalkyl, heterocycloalkyl, aryl or hetaryl.

Suitable monomers of the formula III a) in which X is O are, for example, n-octyl(meth)acrylate, 1,1,3,3-tetramethylbutyl(meth)acrylate, ethylhexyl(meth)acrylate, n-nonyl(meth)acrylate, n-decyl(meth)acrylate, n-undecyl(meth)acrylate, tridecyl(meth)acrylate, myristyl(meth)acrylate, pentadecyl(meth)acrylate, palmityl(meth)acrylate, heptadecyl(meth)acrylate, nonadecyl(meth)acrylate, arrachinyl(meth)acrylate, behenyl(meth)acrylate, lignocerenyl(meth)acrylate, cerotinyl(meth)acrylate, melissinyl(meth)acrylate, palmitoleinyl(meth)acrylate, oleyl(meth)acrylate, linolyl(meth)acrylate, linolenyl(meth)acrylate, stearyl(meth)acrylate, lauryl(meth)acrylate and mixtures thereof.

Suitable monomers of the formula III a) in which X is NR¹⁰ are, for example, n-octyl(meth)acrylamide, 1,1,3,3-tetramethylbutyl(meth)acrylamide, ethylhexyl(meth)acrylamide, n-nonyl(meth)acrylamide, n-decyl(meth)acrylamide, n-undecyl(meth)acrylamide, tridecyl(meth)acrylamide, myristyl(meth)acrylamide, pentadecyl(meth)acrylamide, palmityl(meth)acrylamide, heptadecyl(meth)acrylamide, nonadecyl(meth)acrylamide, arrachinyl(meth)acrylamide, behenyl(meth)acrylamide, lignocerenyl(meth)acrylamide, cerotinyl(meth)acrylamide, melissinyl(meth)acrylamide, palmitoleinyl(meth)acrylamide, oleyl(meth)acrylamide, linolyl(meth)acrylamide, linolenyl(meth)acrylamide, stearyl(meth)acrylamide, lauryl(meth)acrylamide, N-methyl-N-(n-octyl)(meth)acrylamide, N,N,-di(n-octyl)(meth)acrylamide and mixtures thereof.

Suitable monomers of the formula III b) are, for example, n-octyl vinyl ether, 1,1,3,3-tetramethylbutyl vinyl ether, ethylhexyl vinyl ether, n-nonyl vinyl ether, n-decyl vinyl ether, n-undecyl vinyl ether, tridecyl vinyl ether, myristyl vinyl ether, pentadecyl vinyl ether, palmityl vinyl ether, heptadecyl vinyl ether, octadecyl vinyl ether, nonadecyl vinyl ether, arrachinyl vinyl ether, behenyl vinyl ether, lignocerenyl vinyl ether, cerotinyl vinyl ether, melissinyl vinyl ether, palmitoleinyl vinyl ether, oleyl vinyl ether, linolyl vinyl ether, linolenyl vinyl ether, stearyl vinyl ether, lauryl vinyl ether and mixtures thereof.

In the formulae III c) and III d), k is preferably an integer from 1 to 500, in particular 3 to 250. Preferably, l is an integer from 0 to 100.

Preferably, R⁸ in the formula III c) is hydrogen, methyl, ethyl, n-propyl, isopropyl, n-butyl, sec-butyl, tert-butyl, n-pentyl or n-hexyl, in particular hydrogen, methyl or ethyl.

Preferably, R⁹ in the formulae III c) and III d) is n-octyl, 1,1,3,3-tetramethylbutyl, ethylhexyl, n-nonyl, n-decyl, n-undecyl, tridecyl, myristyl, pentadecyl, palmityl, heptadecyl, octadecyl, nonadecyl, arrachinyl, behenyl, lignocerenyl, cerotinyl, melissinyl, palmitoleinyl, oleyl, linolyl, linolenyl, stearyl, lauryl.

Preferably, X in the formula III c) is O or NH.

Suitable polyether acrylates III c) are, for example, the polycondensation products of the abovementioned α,β-ethylenically unsaturated mono- and/or dicarboxylic acids and the acid chlorides, acid amides and anhydrides with polyetherols thereof. Suitable polyetherols can be prepared easily by reacting ethylene oxide, 1,2-propylene oxide and/or epichlorohydrin with a starter molecule, such as water or a short-chain alcohol R⁹—OH. The alkylene oxides can be used individually, alternately one after the other or as a mixture. The polyether acrylates IIIc) can be used on their own or in mixtures for the preparation of the polymers used according to the invention.

Suitable allyl alcohol alkoxylates III d) are, for example, the etherification products of allyl chloride with corresponding polyetherols. Suitable polyetherols can be prepared easily by reacting ethylene oxide, 1,2-propylene oxide and/or epichlorohydrin with a starter alcohol R⁹—OH. The alkylene oxides can be used individually, alternately one after the other or as a mixture. The allyl alcohol alkoxylates III d) can be used on their own or in mixtures for the preparation of the polymers used according to the invention.

Suitable monomers III e) are C₈-C₃₀-, preferably C₈-C₂₂-carboxylic acid vinyl esters. These include, for example, n-octyl vinyl ester, 1,1,3,3-tetramethylbutyl vinyl ester, ethylhexyl vinyl ester, n-nonyl vinyl ester, n-decyl vinyl ester, n-undecyl vinyl ester, tridecyl vinyl ester, myristyl vinyl ester, pentadecyl vinyl ester, palmityl vinyl ester, heptadecyl vinyl ester, octadecyl vinyl ester, nonadecyl vinyl ester, arrachinyl vinyl ester, behenyl vinyl ester, lignocerenyl vinyl ester, cerotinyl vinyl ester, melissinyl vinyl ester, palmitoleinyl vinyl ester, oleyl vinyl ester, linolyl vinyl ester, linolenyl vinyl ester, stearyl vinyl ester, lauryl vinyl ester and mixtures thereof.

Monomer f)

The copolymers A) can additionally comprise, in copolymerized form, at least one monomer f) which is chosen from α,β-ethylenically unsaturated compounds with cationogenic and/or cationic groups that are different from component b).

Preferably, the fraction of monomers f) is 0 to 30% by weight, particularly preferably 0.1 to 20% by weight, in particular 0.5 to 15% by weight, based on the total weight of the compounds used for the polymerization. As detailed above, the copolymers A) preferably have a clear excess of anionogenic/anionic groups or of cationogenic/cationic groups. If, therefore, monomers f) are used, then preferably in amounts such that the copolymer A) has a molar excess of anionogenic/anionic groups compared with cationogenic/cationic groups or a molar excess of cationogenic/cationic groups compared with anionogenic/anionic groups of more than 2:1, preferably at least 2.5:1.

The cationogenic and/or cationic groups of component f) are preferably nitrogen-containing groups, such as primary, secondary and tertiary amino groups, and quaternary ammonium groups. The nitrogen-containing groups are preferably tertiary amino groups or quaternary ammonium groups. Charged cationic groups can be produced from the amine nitrogens either by protonation or by quaternization with acids or with alkylating agents. These include, for example, carboxylic acids, such as lactic acid, or mineral acids, such as phosphoric acid, sulfuric acid and hydrochloric acid, or as alkylating agents C₁-C₄-alkyl halides or sulfates, such as ethyl chloride, ethyl bromide, methyl chloride, methyl bromide, dimethyl sulfate and diethyl sulfate. Protonation or quaternization can generally take place either before or after the polymerization.

Suitable compounds f) are the esters of α,β-ethylenically unsaturated mono- and dicarboxylic acids with amino alcohols. Preferred amino alcohols are C₂-C₁₂-amino alcohols which are C₁-C₈-mono- or -dialkylated on the amine nitrogen. Suitable as acid component of these esters are, for example, acrylic acid, methacrylic acid, fumaric acid, maleic acid, itaconic acid, crotonic acid, maleic anhydride, monobutyl maleate and mixtures thereof. As acid component, preference is given to using acrylic acid, methacrylic acid and mixtures thereof.

Preferred monomers f) are N-tert-butylaminoethyl(meth)acrylate, N,N-dimethylamino-methyl(meth)acrylate, N,N-dimethylaminoethyl(meth)acrylate, N,N-diethylaminoethyl(meth)acrylate, N,N-dimethylaminopropyl(meth)acrylate, N,N-diethylaminopropyl(meth)acrylate and N,N-dimethylaminocyclohexyl(meth)acrylate. Particular preference is given to N-tert-butylaminoethyl(meth)acrylate and N,N-dimethylaminoethyl(meth)acrylate.

Suitable monomers f) are also the amides, different from N-[3-(dimethylamino)propyl]-acrylamide and N-[3-(dimethylamino)propyl]methacrylamide, of the above-mentioned α,β-ethylenically unsaturated mono- and dicarboxylic acids with diamines which have at least one primary or secondary amino group. Preference is given to diamines which have one tertiary amino group and one primary or secondary amino group.

Suitable monomers f) are, for example, N-tert-butylaminoethyl(meth)acrylamide, N-[2-(dimethylamino)ethyl]acrylamide, N-[2-(dimethylamino)ethyl]methacrylamide, N-[4-(dimethylamino)butyl]acrylamide, N-[4-(dimethylamino)butyl]methacrylamide, N-[2-(diethylamino)ethyl]acrylamide, N-[4-(dimethylamino)cyclohexyl]acrylamide and N-[4-(dimethylamino)cyclohexyl]methacrylamide.

Suitable monomers f) are also N,N-diallylamines and N,N-diallyl-N-alkylamines and acid addition salts and quaternization products thereof. Alkyl here is preferably C₁-C₂₄-alkyl. Preference is given to N,N-diallyl-N-methylamine and N,N-diallyl-N,N-dimethylammonium compounds, such as, for example, the chlorides and bromides. These include, in particular, N,N-diallyl-N-methylamine.

Suitable monomers f) are also vinyl- and allyl-substituted nitrogen heterocycles different from component b), such as 2- and 4-vinylpyridine, 2- and 4-allylpyridine, and the salts thereof.

Monomer g)

The copolymers A) according to the invention can additionally comprise, in copolymerized form, at least one monomer g) which is different from the components a) to f) and is copolymerizable therewith.

Preferably, the fraction of monomers is up to 40% by weight, based on the total weight of the compounds used for the polymerization. A suitable use amount for additional monomers g) is in a range from 0.1 to 25% by weight, in particular 0.5 to 20% by weight, based on the total weight of the compounds used for the polymerization.

Preferably, the component g) is chosen from esters of α,β-ethylenically unsaturated mono- and dicarboxylic acids with C₁-C₇-alkanols, esters of α,β-ethylenically unsaturated mono- and dicarboxylic acids with C₂-C₃₀-diols, amides of α,β-ethylenically unsaturated mono- and dicarboxylic acids with C₂-C₃₀-amino alcohols which have a primary or secondary amino group, polyether acrylates different from III c), esters of vinyl alcohol and allyl alcohol with C₁-C₇-monocarboxylic acids, C₁-C₇-alkyl vinyl ethers, allyl alcohol alkoxylates different from III d), vinylaromatics, vinyl halides, vinylidene halides, C₂-C₈-monoolefins, nonaromatic hydrocarbons with at least two conjugated double bonds and mixtures thereof.

Suitable esters of α,β-ethylenically unsaturated mono- and dicarboxylic acids with C₁-C₇-alkanols are, for example, methyl(meth)acrylate, methyl ethacrylate, ethyl(meth)acrylate, ethyl ethacrylate, n-butyl(meth)acrylate, tert-butyl(meth)acrylate, tert-butyl ethacrylate, n-pentyl(meth)acrylate, n-hexyl(meth)acrylate, n-heptyl(meth)acrylate, etc. Preferred monomers g) are the esters of α,β-ethylenically unsaturated mono- and dicarboxylic acids with C₁-C₃-alkanols, in particular methyl methacrylate.

Suitable additional monomers g) are also 2-hydroxyethyl acrylate, 2-hydroxyethyl methacrylate, 2-hydroxyethyl ethacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, 4-hydroxybutyl acrylate, 4-hydroxybutyl methacrylate, 6-hydroxyhexyl acrylate, 6-hydroxyhexyl methacrylate, 3-hydroxy-2-ethylhexyl acrylate and 3-hydroxy-2-ethylhexyl methacrylate.

Suitable additional monomers g) are also 2-hydroxyethylacrylamide, 2-hydroxy-ethylmethacrylamide, 2-hydroxyethylethacrylamide, 2-hydroxypropylacrylamide, 2-hydroxypropylmethacrylamide, 3-hydroxypropylacrylamide, 3-hydroxypropylmeth-acrylamide, 3-hydroxybutylacrylamide, 3-hydroxybutylmethacrylamide, 4-hydroxybutyl-acrylamide, 4-hydroxybutylmethacrylamide, 6-hydroxyhexylacrylamide, 6-hydroxy-hexylmethacrylamide, 3-hydroxy-2-ethylhexylacrylamide and 3-hydroxy-2-ethylhexyl-methacrylamide.

Also suitable as component g) are polyether acrylates of the general formula III c*) different from compounds of the formula III c) and allyl alcohol alkoxylates of the formula III d*) different from compounds of the formula III d)

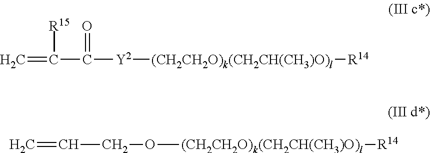

in which
the order of the alkylene oxide units is arbitrary,
k and l, independently of one another, are an integer from 0 to 1000, where the sum of k and l is at least 5,
R¹⁴ is hydrogen, C₁-C₇-alkyl or C₅-C₈-cycloalkyl,
R¹⁵ is hydrogen or C₁-C₈-alkyl,
Y² is O or NR⁶, where R⁶ is hydrogen, C₁-C₃₀-alkyl or C₅-C₈-cycloalkyl.

In the formulae III c*) and III d*) k is preferably an integer from 1 to 500, in particular 3 to 250. Preferably, l is an integer from 0 to 100.

Preferably, R¹⁵ in the formula III c*) is hydrogen, methyl, ethyl, n-propyl, isopropyl, n-butyl, sec-butyl, tert-butyl, n-pentyl or n-hexyl, in particular hydrogen, methyl or ethyl.

Preferably, R¹⁴ in the formulae III c*) and III d*) is hydrogen, methyl, ethyl, n-propyl, isopropyl, n-butyl, sec-butyl, n-pentyl, n-hexyl.

Preferably, Y² in the formula III c*) is O or NH.

Suitable polyether acrylates III c*) are, for example, the polycondensation products of the abovementioned α,β-ethylenically unsaturated mono- and/or dicarboxylic acids and the acid chlorides, acid amides and anhydrides with polyetherols thereof. Suitable polyetherols can be prepared easily by reacting ethylene oxide, 1,2-propylene oxide and/or epichlorohydrin with a starter molecule, such as water or a short-chain alcohol R¹⁴—OH. The alkylene oxides can be used individually, alternately one after the other or as a mixture. The polyether acrylates III c*) can be used on their own or in mixtures for the preparation of the polymers used according to the invention.

Suitable allyl alcohol alkoxylates III d*) are, for example, the etherification products of allyl chloride with corresponding polyetherols. Suitable polyetherols can be prepared easily by reacting ethylene oxide, 1,2-propylene oxide and/or epichlorohydrin with water or a starter alcohol R⁹*—OH. The alkylene oxides can be used individually, alternately one after the other or as a mixture. The allyl alcohol alkoxylates III d*) can be used on their own or in mixtures for the preparation of the polymers used according to the invention.

Suitable polyether acrylates g) are also urethane (meth)acrylates with alkylene oxide groups. Such compounds are described in DE 198 38 851 (component e2)), which is hereby incorporated in its entirety by reference.

Suitable additional monomers g) are also vinyl acetate, vinyl propionate, vinyl butyrate and mixtures thereof.

Suitable additional monomers g) are also ethylene, propylene, isobutylene, butadiene, styrene, α-methylstyrene, acrylonitrile, methacrylonitrile, vinyl chloride, vinylidene chloride, vinyl fluoride, vinylidene fluoride and mixtures thereof.

The abovementioned additional monomers g) can in each case be used individually or in the form of any desired mixtures.

Preference is given to a copolymer A) which comprises, in copolymerized form,
2 to 96% by weight of at least one compound a) having a free-radically polymerizable α,β-ethylenically unsaturated double bond and at least one anionogenic and/or anionic group per molecule, preferably acrylic acid and/or methacrylic acid,
2 to 96% by weight of at least one compound b) which is chosen from N-vinylimidazole compounds, N-[3-(dimethylamino)propyl]acrylamide, N-[3-(dimethylamino)propyl]methacrylamide and mixtures thereof,
0.05 to 5% by weight of at least one crosslinker c), preferably ethylene glycol di(meth)acrylate and/or pentaerythritol triallyl ether,
0 to 95% by weight of at least one amide-containing monomer d), preferably vinylpyrrolidone and/or vinylcaprolactam,
0 to 40% by weight of at least one compound e) which is preferably chosen from C₈-C₂₂-(meth)acrylates, C₈-C₂₂-alkyl vinyl ethers, polyether (meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
0 to 40% by weight of at least one monomer g) which is preferably chosen from C₁-C₆-(meth)acrylates, in particular methyl(meth)acrylate, ethyl(meth)acrylate, n-butyl(meth)acrylate and mixtures thereof.

Instead of or in addition to at least one compound b), the copolymers A) can comprise, in copolymerized form, at least one compound f) having a free-radically polymerizable α,β-ethylenically unsaturated double bond and at least one cationogenic and/or cationic group per molecule. Preferably, the monomers with cationogenic/cationic groups are exclusively chosen from the compounds b).

The ampholytic copolymers A) are preferably obtainable by free-radical copolymerization of
at least 2% by weight, based on the total weight of the monomers used for the polymerization, of at least one monomer pair of at least one N-vinylimidazole compound b) and acrylic acid and/or methacrylic acid,
at least 1% by weight, based on the total weight of the monomers used for the polymerization, of at least one monomer with an anionogenic or anionic group or at least one monomer with a cationogenic or cationic group, in each case chosen from the monomers a), b) or f),
0.05 to 5% by weight, based on the total weight of the monomers used for the polymerization, of at least one free-radically polymerizable crosslinking compound c),
0 to 95% by weight of at least one monomer d) containing amide groups,
0 to 40% by weight, based on the total weight of the monomers used for the polymerization, of at least one hydrophobic monomer e),
0 to 40% by weight, based on the total weight of the monomers used for the polymerization, of at least one monomer g).

A specific embodiment of the copolymers A) according to the invention covers anionically ampholytic copolymers. These comprise, as component a), preferably acrylic acid, methacrylic acid or a mixture thereof. The component b) is preferably chosen from N-vinylimidazole, N-[3-(dimethylamino)propyl]acrylamide, N-[3-(dimethylamino)propyl]methacrylamide and mixtures thereof. A preferred embodiment covers copolymers A) which have a molar excess of anionogenic/anionic groups compared with cationogenic/cationic groups of more than 1:1, preferably at least 1.2:1, in particular at least 1.4:1.

Preferred anionically ampholytic copolymers A) are obtainable by free-radical copolymerization of
methacrylic acid and/or acrylic acid a),
at least one compound b) chosen from N-vinylimidazole, N-[3-(dimethylamino)propyl]acrylamide, N-[3-(dimethylamino)propyl]methacrylamide and mixtures thereof,
at least one crosslinker c), preferably ethylene glycol di(meth)acrylate and/or pentaerythritol triallyl ether,

where the molar ratio of anionogenic/anionic groups to cationogenic/cationic groups is at least 1:1.

Preferred anionically ampholytic copolymers A) are obtainable by free-radical copolymerization of
methacrylic acid and/or acrylic acid a),
at least one compound b) chosen from N-vinylimidazole, N-[3-(dimethylamino)propyl]acrylamide, N-[3-(dimethylamino)propyl]methacrylamide and mixtures thereof,
at least one crosslinker c), preferably ethylene glycol di(meth)acrylate and/or pentaerythritol triallyl ether,
3 to 35% by weight, based on the total weight of the monomers used for the polymerization, of at least one monomer g) which is preferably chosen from C₁-C₆-(meth)acrylates,

where the molar ratio of anionogenic/anionic groups to cationogenic/cationic groups is at least 1.2:1.

Preferred anionically ampholytic copolymers A) are obtainable by free-radical copolymerization of
methacrylic acid and/or acrylic acid a),
at least one compound b) chosen from N-vinylimidazole, N-[3-(dimethylamino)propyl]acrylamide, N-[3-(dimethylamino)propyl]methacrylamide and mixtures thereof,
at least one crosslinker c), preferably ethylene glycol di(meth)acrylate and/or pentaerythritol triallyl ether,
0.1 to 10% by weight, based on the total weight of the monomers used for the polymerization, of at least one compound e) which is preferably chosen from C₈-C₂₂-(meth)acrylates, C₈-C₂₂-alkyl vinyl ethers, polyether (meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,

where the molar ratio of anionogenic/anionic groups to cationogenic/cationic groups is at least 1.4:1.

In the case of the three last-mentioned anionically ampholytic copolymers, up to 60% by weight of the component a), based on the total weight of the monomers a), can be replaced by at least one monomer d), preferably vinylpyrrolidone and/or vinylcaprolactam.

Preference is given to anionically ampholytic copolymers A) for whose preparation at least some of the monomers a) and b) are used in the form of a monomer pair.

Particularly preferred anionic ampholytic copolymers A) are obtainable by free-radical copolymerization of
at least 2% by weight, based on the total weight of the monomers used for the polymerization, of at least one monomer pair of N-vinylimidazole and acrylic acid and/or methacrylic acid,
5 to 70% by weight of methacrylic acid and/or acrylic acid,
0.1 to 2% by weight of at least one crosslinker c), preferably ethylene glycol di(meth)acrylate and/or pentaerythritol triallyl ether,
20 to 95% by weight of vinylpyrrolidone,
0 to 40% by weight of at least one further monomer which is chosen from methyl(meth)acrylate, ethyl(meth)acrylate, n-butyl(meth)acrylate, C₈-C₂₂-(meth)-acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof.

Instead of or in addition to vinylpyrrolidone, it is also possible to use vinylcaprolactam. Instead of or in addition to vinylimidazole, it is also possible to use N-[3-(dimethylamino)propyl]acrylamide and/or N-[3-(dimethylamino)propyl]methacrylamide.

Particularly preferred anionic ampholytic copolymers A) are also obtainable by free-radical copolymerization of
at least 5% by weight, based on the total weight of the monomers used for the polymerization, of at least one monomer pair of N-vinylimidazole and acrylic acid and/or methacrylic acid,
5 to 70% by weight of methacrylic acid and/or acrylic acid,
0.1 to 2% by weight of at least one crosslinker c), preferably ethylene glycol di(meth)acrylate and/or pentaerythritol triallyl ether,
20 to 85% by weight of vinylpyrrolidone,
5 to 40% by weight of at least one further monomer which is chosen from C₁-C₆-(meth)acrylates, in particular methyl(meth)acrylate, ethyl(meth)acrylate, n-butyl(meth)acrylate and mixtures thereof.

Instead of or in addition to vinylpyrrolidone, it is also possible to use vinylcaprolactam. Instead of or in addition to vinylimidazole, it is also possible to use N-[3-(dimethylamino)propyl]acrylamide and/or N-[3-(dimethylamino)propyl]methacrylamide.

Particularly preferred anionic ampholytic copolymers A) are also obtainable by free-radical copolymerization of
at least 5% by weight, based on the total weight of the monomers used for the polymerization, of at least one monomer pair of N-vinylimidazole and acrylic acid and/or methacrylic acid,
5 to 70% by weight of methacrylic acid and/or acrylic acid,
0.1 to 2% by weight of at least one crosslinker c), preferably ethylene glycol di(meth)acrylate and/or pentaerythritol triallyl ether,
20 to 85% by weight of vinylpyrrolidone,
1 to 20% by weight of at least one further monomer which is chosen from C₈-C₃₀-(meth)acrylates, polyether(meth)acrylates terminated with C₈-C₃₀-alkyl groups, and mixtures thereof, in particular from stearyl methacrylate, polyethylene glycol(meth)acrylates terminated with C₁₈-C₂₂-alkyl groups, and mixtures thereof.

Instead of or in addition to vinylpyrrolidone, it is also possible to use vinylcaprolactam. Instead of or in addition to vinylimidazole, it is also possible to use N-[3-(dimethylamino)propyl]acrylamide and/or N-[3-(dimethylamino)propyl]methacrylamide.

In a specific embodiment, all of the abovementioned anionic ampholytic copolymers A) which comprise at least 5% by weight of at least one vinylimidazole compound in copolymerized form are subjected to partial or complete quaternization. Suitable quaternizing agents are those specified below.

A further specific embodiment of the copolymers A) according to the invention covers cationically ampholytic copolymers. These comprise, as component a), preferably acrylic acid, methacrylic acid or a mixture thereof. Preferred components b) are N-vinylimidazole and mixtures of N-vinylimidazole with N-[3-(dimethylamino)propyl]-acrylamide and/or N-[3-(dimethylamino)propyl]methacrylamide.

Preferred cationically ampholytic copolymers A) are obtainable by free-radical copolymerization of
methacrylic acid and/or acrylic acid a),
N-vinylimidazole b),
at least one crosslinker c), preferably ethylene glycol di(meth)acrylate and/or pentaerythritol triallyl ether,

where the molar ratio of cationogenic/cationic groups to anionogenic/anionic groups is at least 6:1.

Preferred cationically ampholytic copolymers A) are obtainable by free-radical copolymerization of
methacrylic acid and/or acrylic acid a),
N-vinylimidazole b),
at least one crosslinker c), preferably ethylene glycol di(meth)acrylate and/or pentaerythritol triallyl ether,
3 to 35% by weight, based on the total weight of the monomers used for the polymerization, of at least one monomer g) which is preferably chosen from C₁-C₈-(meth)acrylates,

where the molar ratio of cationogenic/cationic groups to anionogenic/anionic groups is at least 6:1.

Preferred cationically ampholytic copolymers A) are obtainable by free-radical copolymerization of
methacrylic acid and/or acrylic acid a),
N-vinylimidazole b),
at least one crosslinker c), preferably ethylene glycol di(meth)acrylate and/or pentaerythritol triallyl ether,
0.1 to 10% by weight of at least one compound e) which is preferably chosen from C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,

where the molar ratio of cationogenic/cationic groups to anionogenic/anionic groups is at least 6:1.

In the case of the three last-mentioned cationically ampholytic copolymers, up to 50% by weight of the N-vinylimidazole a2), based on the total weight of the components a2), can be replaced by another monomer, preferably N-[3-(dimethylamino)propyl]acryl-amide and/or N-[3-(dimethylamino)propyl]methacrylamide.

Preference is also given to cationically ampholytic copolymers A) for whose preparation at least some of the monomers a1) and a2) are used in the form of a monomer pair.

Particularly preferred cationic ampholytic copolymers A) are obtainable by free-radical copolymerization of
at least 2% by weight, based on the total weight of the monomers used for the polymerization, of at least one monomer pair of N-vinylimidazole and acrylic acid and/or methacrylic acid,
3 to 70% by weight of at least one monomer with a cationogenic or cationic group, preferably chosen from vinylimidazole compounds, N-[3-(dimethylamino)-propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker c), preferably ethylene glycol di(meth)acrylate and/or pentaerythritol triallyl ether,
0 to 95% by weight, preferably 20 to 95% by weight of vinylpyrrolidone and/or vinylcaprolactam,
0 to 40% by weight of at least one further monomer which is chosen from methyl(meth)acrylate, ethyl(meth)acrylate, n-butyl(meth)acrylate, C₈-C₂₂-(meth)-acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof.

Particularly preferred cationic ampholytic copolymers A) are also obtainable by free-radical copolymerization of
at least 2% by weight, based on the total weight of the monomers used for the polymerization, of at least one monomer pair of N-vinylimidazole and acrylic acid and/or methacrylic acid,
3 to 50% by weight of at least one monomer with a cationogenic or cationic group, preferably chosen from vinylimidazole compounds, N-[3-(dimethylamino)-propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker c), preferably ethylene glycol di(meth)acrylate and/or pentaerythritol triallyl ether,
20 to 95% by weight of vinylpyrrolidone and/or vinylcaprolactam d),
5 to 40% by weight of at least one further monomer which is chosen from C₁-C₆-(meth)acrylates, in particular methyl(meth)acrylate, ethyl(meth)acrylate, n-butyl(meth)acrylate and mixtures thereof.

Particularly preferred cationic ampholytic copolymers A) are also obtainable by free-radical copolymerization of
at least 5% by weight, based on the total weight of the monomers used for the polymerization, of at least one monomer pair of N-vinylimidazole and acrylic acid and/or methacrylic acid,
3 to 70% by weight of at least one monomer with a cationogenic or cationic group, preferably chosen from vinylimidazole compounds, N-[3-(dimethylamino)-propyl]acrylamide, N-[3-(dimethylamino)propyl]methacrylamide, N,N-dimethyl-laminoethyl(meth)acrylate, N,N-dimethylaminopropyl(meth)acrylate and mixtures thereof,
0.1 to 2% by weight of at least one crosslinker c), preferably ethylene glycol di(meth)acrylate and/or pentaerythritol triallyl ether,
20 to 85% by weight of vinylpyrrolidone and/or vinylcaprolactam d),
1 to 20% by weight of at least one further monomer which is chosen from C₈-C₃₀-(meth)acrylates, polyether(meth)acrylates terminated with C₈-C₃₀-alkyl groups, and mixtures thereof, in particular from stearyl methacrylate, polyethylene glycol(meth)acrylates terminated with C₁₈-C₂₂-alkyl groups, and mixtures thereof.

In a specific embodiment, all of the abovementioned cationic ampholytic copolymers A) are subjected to partial or complete quaternization. Suitable quaternizing agents are those specified below.

In a specific embodiment, the free-radical copolymerization of the above-mentioned components a) to c) and, if present, d) to g) is carried out in the presence of at least one polyether-containing compound which has no copolymerizable double bond. Here, specific ampholytic copolymers having advantageous properties are obtained. This can be attributed, for example, to the effect of the polyether component as protective colloid or emulsifier. This can, for example, also result from an at least partial grafting onto the polyether component as graft base. However, mechanisms other than grafting are also conceivable. The ampholytic copolymers according to the invention comprise, quite generally, the process products of the free-radical copolymerization, which are understood as meaning, for example, pure graft polymers, mixtures of graft polymers with ungrafted compounds of the polyether component, and any desired mixtures.

Preferably, the amount of polyether component used (if present) is 0.1 to 50% by weight, particularly preferably 1 to 25% by weight, based on the total weight of the components used for the polymerization.

Suitable polyether-containing compounds are, for example, water-soluble or water-dispersible nonionic polymers which have alkylene oxide repeat units. The fraction of alkylene oxide repeat units is preferably at least 30% by weight, based on the total weight of the compound. Suitable polyether-containing compounds are, for example, polyalkylene glycols, as are usually also used as nonionic surfactants. Suitable polyalkylene glycols generally have a number-average molecular weight in the range from about 150 to 100 000, preferably 300 to 50 000, particularly preferably 500 to 40 000. Suitable polyalkylene glycols are, for example, polyethylene glycols, polypropylene glycols, polytetrahydrofurans and alkylene oxide copolymers. Suitable alkylene oxides for the preparation of alkylene oxide copolymers are, for example, ethylene oxide, propylene oxide, epichlorohydrin, 1,2- and 2,3-butylene oxide. The alkylene oxide copolymers can comprise the copolymerized alkylene oxide units in random distribution or in the form of blocks. Advantageously, homopolymers of ethylene oxide or copolymers which comprise ethylene oxide are used. Preferably, the fraction of repeat units derived from ethylene oxide is 40 to 99% by weight. Of suitability are, for example, copolymers of ethylene oxide and propylene oxide, copolymers of ethylene oxide and butylene oxide, and copolymers of ethylene oxide, propylene oxide and at least one butylene oxide.

The copolymers A1) are prepared in accordance with customary processes known to the person skilled in the art, e.g. by solution polymerization, precipitation polymerization, suspension polymerization or emulsion polymerization. The W/W polymerization in water with a suitable displacing agent, e.g. a salt, such as NaCl, is also suitable.

Preferred solvents for the solution polymerization are aqueous solvents, such as water and mixtures of water with water-miscible solvents, for example alcohols, such as methanol, ethanol, n-propanol, isopropanol, n-butanol, sec-butanol, tert-butanol, n-hexanol and cyclohexanol, and glycols, such as ethylene glycol, propylene glycol and butylene glycol, and the methyl or ethyl ethers of the dihydric alcohols, diethylene glycol, triethylene glycol, polyethylene glycols with number-average molecular weights up to about 3000, glycerol and dioxane. Particular preference is given to the polymerization in water or a water/alcohol mixture, for example in a water/ethanol mixture. The polymerization temperatures in the case of solution polymerization are preferably in a range from about 30 to 120° C., particularly preferably 40 to 100° C.

The ampholytic copolymers A) are particularly preferably prepared by precipitation polymerization.

The precipitation polymerization preferably takes place in an anhydrous, aprotic solvent or solvent mixture, preferably in ethyl acetate and/or n-butyl acetate. The precipitation polymerization preferably takes place at a temperature in the range from 70 to 140° C., preferably 75 to 100° C., in particular from 80 to 95° C. The resulting polymer particles precipitate out of the reaction solution and can be isolated by customary methods, such as filtration using subatmospheric pressure. For the precipitation polymerization it is possible to use surface-active, polymeric compounds, preferably based on polysiloxanes. In the case of precipitation polymerization, the polymers obtained usually have higher molecular weights than those in the case of solution polymerization.

The polymerization is usually carried out under atmospheric pressure, although it can also proceed under reduced or elevated pressure. A suitable pressure range is between 1 and 5 bar.

To prepare the polymers, the monomers can be polymerized with the help of initiators which form free radicals.

Initiators for the free-radical polymerization which can be used are the peroxo and/or azo compounds customary for this purpose, for example alkali metal or ammonium peroxydisulfates, diacetyl peroxide, dibenzoyl peroxide, succinyl peroxide, di-tert-butyl peroxide, tert-butyl perbenzoate, tert-butyl perpivalate, tert-butyl peroxy-2-ethyl-hexanoate, tert-butyl permaleate, cumene hydroperoxide, diisopropyl peroxydicar-bamate, bis(o-toloyl) peroxide, didecanoyl peroxide, dioctanoyl peroxide, dilauroyl peroxide, tert-butyl perisobutyrate, tert-butyl peracetate, di-tert-amyl peroxide, tert-butyl hydroperoxide, 2,2′-azobisisobutyronitrile, azobis(2-amidinopropane) dihydrochloride or 2-2′-azobis(2-methylbutyronitrile). Also suitable are initiator mixtures or redox initiator systems, such as, for example, ascorbic acid/iron(II) sulfate/sodium peroxodisulfate, tert-butyl hydroperoxide/sodium disulfite, tert-butyl hydroperoxide/sodium hydroxymethanesulfinate, H₂O₂/Cu^{l}.

In a specific embodiment, for the preparation of the copolymers according to the invention, use is made of at least two free-radical initiators which permit an essentially independent initiation in at least two phases. In this case, copolymers with particularly low residual monomer contents can be achieved.

Preferably, for the copolymerization, at least two initiators are used whose decomposition temperatures are different from one another by at least 10° C. Within the scope of the invention, the decomposition temperature is defined as the temperature at which 50% of the molecules decompose into free radicals within 2.5 hours. Preferably, the copolymerization in the case of this procedure takes place until conclusion of the precipitation of the copolymer at a temperature greater than or equal to the lower decomposition temperature and less than the higher decomposition temperature, and, after the precipitation, a further reaction takes place at a temperature greater than or equal to the higher decomposition temperature.

Preferably, the method according to the invention comprises a first polymerization phase at a first polymerization temperature and a second polymerization phase at a second polymerization temperature above the first polymerization temperature, where for the polymerization at least two initiators are used whose half-lives at the first polymerization temperature differ in such a way that at least one of these initiators decomposes into free radicals during the first polymerization phase and at least one of these initiators essentially does not decompose into free radicals during the first polymerization phase and decomposes into free radicals during the second polymerization phase. Preferably, in the case of this procedure, the second polymerization phase starts essentially after precipitation of the copolymer. “Essentially” after precipitation of the copolymer is understood as meaning that the copolymer is present preferably to at least 80% by weight, preferably to at least 90% by weight, in particular at least 95% by weight, based on the total weight of the copolymer, in precipitated form.

The half-life of an initiator can be determined by customary methods known to the person skilled in the art, as described, for example, in the publication “Initiators for high polymers”, Akzo Nobel, No. 10737. The half-life of the first polymerization initiator at the first polymerization temperature and of the second polymerization initiator at the second polymerization temperature is preferably in a range from about 1 minute to 3 hours, particularly preferably 5 minutes to 2.5 hours. If desired, shorter half-lives of, for example, 1 second to 1 minute, or half-lives longer than 3 hours can be used provided it is ensured that the initiator(s) decomposing at the higher temperature essentially decompose(s) into free radicals during the second polymerization phase.

In addition to the first and second polymerization phase, further polymerization phases can be applied at polymerization temperatures different therefrom. Thus, it is, for example, possible to carry out a first polymerization phase at a first polymerization temperature which is chosen so that a controlled polymerization (i.e. e.g. avoiding an undesired temperature increase as a result of the heat of reaction, an excessively high reaction rate, etc.) takes place. An afterpolymerization can then, for example, follow at a temperature which is above the first and below the second polymerization temperature and is chosen so that the initiator(s) decomposing at the higher temperature does not essentially decompose into free radicals. Following completion of this afterpolymerization, to which, if desired, the initiator decomposing at the lower temperature and/or another initiator decomposing under the conditions of the afterpolymerization can also be added, the second polymerization phase can then follow.

Preferably, the initiator system used comprises at least two initiators whose decomposition temperatures differ from one another by at least 15° C.

The initiator decomposing at the lower temperature preferably has a decomposition temperature of from 50 to 100° C.

The initiator decomposing at the higher temperature preferably has a decomposition temperature of from 80 to 150° C.

Preferably, the initiator decomposing at the higher temperature is initially introduced at the start of the copolymerization or is added before or during the precipitation of the copolymer.

Preferably, the initiator decomposing at the higher temperature is initially introduced at the start of the copolymerization or is added before the precipitation of the copolymer.

In a preferred initiator combination, the initiator decomposing at the lower temperature is Trigonox® EHP (bis(2-ethylhexyl)peroxydicarbonate, CAS-No. 16111-62-9) and the initiator decomposing at the higher temperature is chosen from tert-butyl peroxypivalate (e.g. Luperox 11 M75 from Atochem), tert-butyl peroctoate, lauroyl peroxide (LPO, CAS-No. 105-74-8) or 2,5-dimethyl-2,5-bis(t-butylperoxy)hexane (Trigonox® 101).

To achieve the purest polymers possible, the polymers can, for example, be subjected to a washing step with a suitable solvent, e.g. the solvent also used for the polymerization.

The anionogenic groups (acid groups) of the copolymers A) can be partially or completely neutralized with a base. Bases which can be used for the neutralization of the polymers are alkali metal bases such as sodium hydroxide solution, potassium hydroxide solution, sodium carbonate, sodium hydrogencarbonate, potassium carbonate or potassium hydrogencarbonate and alkaline earth metal bases, such as calcium hydroxide, calcium oxide, magnesium hydroxide or magnesium carbonate, and amines. Suitable amines are, for example, C₁-C₆-alkylamines, preferably n-propylamine and n-butylamine, dialkylamines, preferably diethylpropylamine and dipropylmethyl-amine, trialkylamines, preferably triethylamine and triisopropylamine. Preference is given to amino alcohols, e.g. trialkanolamines, such as triethanolamine, alkyl-dialkanolamines, such as methyl- or ethyldiethanolamine and dialkylalkanolamines, such as dimethylethanolamine, and 2-amino-2-methyl-1-propanol. For use in hair-treatment compositions in particular, NaOH, KOH, 2-amino-2-methyl-1-propanol, 2-amino-2-ethylpropane-1,3-diol, diethylaminopropylamine and triisopropanolamine have proven useful for neutralizing the polymers comprising acid groups. The neutralization of the acid groups can also be carried out using mixtures of two or more bases, e.g. mixtures of sodium hydroxide solution and triisopropanolamine. Depending on the intended use, the neutralization can be carried out partially or completely.

Charged cationic groups can be produced from the present cationogenic nitrogen-containing groups either by protonation, e.g. with mono- or polybasic carboxylic acids, such as lactic acid or tartaric acid, or with mineral acids, such as phosphoric acid, sulfuric acid and hydrochloric acid, or by quaternization, e.g. with alkylating agents, such as C₁-C₄-alkyl halides or sulfates. Examples of such alkylating agents are ethyl chloride, ethyl bromide, methyl chloride, methyl bromide, dimethyl sulfate and diethyl sulfate.

If the copolymers A) according to the invention are to be both quaternized and also neutralized, then the quaternization is preferably carried out first, followed by the neutralization.

The ampholytic copolymers A) according to the invention are advantageously suitable for modifying the rheological properties of aqueous active substance or effect substance compositions. These may, quite generally, be for example cosmetic compositions, pharmaceutical compositions, hygiene products, paints, compositions for the paper industry and the textile industry. In a preferred embodiment, the compositions comprise at least one water-soluble or at least water-dispersible active substance or effect substance. The copolymers A) according to the invention are of course also suitable for modifying the rheological properties of compositions which comprise at least one water-insoluble (hydrophobic) active substance or effect substance.

For the purposes of the present invention, “modifying rheological properties” is understood in the wide sense. The copolymers A) used according to the invention are generally suitable for thickening the consistency of aqueous compositions within a wide range. Depending on the basic consistency of the liquid compositions, flow properties from low viscosity to solid (in the sense of “no longer flowable”) can generally be achieved depending on the amount of the copolymer A) used. “Modifying rheological properties” is therefore understood, inter alia, as meaning the increase in the viscosity of liquids, the improvement in the thixotropy properties of gels, the solidification of gels and waxes etc. The compositions according to the invention are preferably suitable for the formulation of aqueous cosmetic and pharmaceutical products. Preferably, the compositions of the copolymers A) are generally clear. Thus, formulations, in particular cosmetic formulations, can advantageously be colored without impairment by the intrinsic color of the compositions. Furthermore, the compositions can be formulated in the form of opaque to clear gels.

The ampholytic copolymers A) according to the invention are specifically suitable as rheology modifiers with properties which can be controlled via the pH. Thus, for example, the abovementioned anionic ampholytic copolymers A) are suitable as pH-switchable thickeners for a pH range of greater than or equal to 6. The abovementioned cationic ampholytic copolymers A) are suitable as pH-switchable thickeners for a pH range of less than or equal to 6.5. Quaternized ampholytic copolymers A) which essentially have no protonatable groups are suitable as rheology modifiers in a pH range from about 2 to 10 irrespective of the pH.

The ampholytic copolymers A) according to the invention are also specifically suitable as rheology modifiers for salt-containing compositions.

Advantageously, the ampholytic copolymers A) according to the invention also act as film-forming and/or conditioning rheology modifiers. They are thus suitable specifically for cosmetic and dermatological compositions, specifically in hair-setting compositions as “setting thickeners” and in hair care compositions as “conditioning thickeners”.

The ampholytic copolymers A) are suitable both for the preparation of homogeneous-phase aqueous compositions, and also for the formulation of heterogeneous-phase compositions which additionally comprise at least one water-insoluble (hydrophobic) liquid or solid compound. “Homogeneous-phase compositions” have only a single phase irrespective of the number of their constituents. “Heterogeneous-phase compositions” are disperse systems of two or more immiscible components. These include solid/liquid, liquid/liquid and solid/liquid/liquid compositions, such as dispersions and emulsions, e.g. O/W and W/O formulations which have at least one of the oil or fat components described in more detail below and water as immiscible phases. In principle, the copolymers A) can be used either in the water phase or in the oil phase. In general, heterogeneous-phase liquid/liquid compositions comprise the copolymers A) essentially in the water phase.

The copolymers A) according to the invention are also suitable as solubilizer for essentially water-insoluble compounds. The invention thus further provides the use of an ampholytic copolymer A), as defined above, as solubilizer for the preparation of aqueous formulations of active substances and effect substances which have a solubility in water at 25° C. and 1013 mbar below 10 g/l.

Furthermore, it has been found that the copolymers A) according to the invention are advantageously suitable as protective colloid. The invention thus further provides the use of an ampholytic copolymer A), as defined above, as protective colloid during free-radical aqueous emulsion polymerization.

On the basis of the abovementioned properties, the copolymers A) according to the invention are very generally suitable for the preparation of active substance or effect substance compositions comprising
A) at least one ampholytic copolymer, as defined above,
B) at least one active substance or effect substance and
C) if appropriate at least one further active substance or auxiliary different from A) and B).

Active substances for cosmetics, medicaments, hygiene compositions, textile-treatment compositions etc., i.e. substances which generally develop an effect even at low concentration, e.g. a cosmetic effect on skin and/or hair, a pharmacological effect within an organism, a cleaning and/or disinfectant effect, a modification of a textile, e.g. a crease-free finishing, and also effect substances, which impart a certain property to living beings or inanimate substrates, for example color pigments for make-up or emulsion paints, are often formulated and applied in the form of aqueous active substance or effect substance compositions.

The active substance and effect substance compositions comprise the polymer component A) preferably in a fraction of from about 0.001 to 50% by weight, particularly preferably 0.01 to 30% by weight, in particular 0.1 to 20% by weight, based on the total weight of the composition.

The components B) and C) are chosen according to the desired field of use of the composition. Besides components which are typical of the field of use (e.g. certain pharmaceutical active substances), they are chosen from carriers, excipients, emulsifiers, surfactants, preservatives, fragrances, thickeners different from component A), polymers, gel formers, dyes, pigments, photoprotective agents, consistency regulators, antioxidants, antifoams, antistats, resins, solvents, solubility promoters, neutralizing agents, stabilizers, sterilizing agents, propellants, drying agents, opacifiers, etc.

The compositions preferably have a carrier component C) which is chosen from water, hydrophilic components, hydrophobic components and mixtures thereof.

Suitable hydrophilic carriers C) are, for example, mono-, di- or polyhydric alcohols having preferably 1 to 8 carbon atoms, such as ethanol, n-propanol, isopropanol, propylene glycol, glycerol, sorbitol, etc.

Suitable hydrophobic carriers C) are preferably chosen from
i) oils, fats, waxes,
ii) esters of C₆-C₃₀-monocarboxylic acids with mono-, di- or trihydric alcohols which are different from iii),
iii) saturated acyclic and cyclic hydrocarbons,
iv) fatty acids,
v) fatty alcohols,
vi) propellant gases,

and mixtures thereof.

Suitable silicone oils C) are, for example, linear polydimethylsiloxanes, poly(methylphenylsiloxanes), cyclic siloxanes and mixtures thereof. The number-average molecular weight of the polydimethylsiloxanes and poly(methylphenylsiloxanes) is preferably in a range from about 1000 to 150 000 g/mol.

Preferred cyclic siloxanes have 4- to 8-membered rings. Suitable cyclic siloxanes are commercially available, for example, under the name cyclomethicone.

Preferred oil and fat components C) are chosen from paraffin and paraffin oils; vaseline; natural fats and oils, such as castor oil, soya oil, peanut oil, olive oil, sunflower oil, sesame oil, avocado oil, cocoa butter, almond oil, peach kernel oil, ricinus oil, cod-liver oil, pig fat, spermaceti, spermaceti oil, sperm oil, wheatgerm oil, macadamia nut oil, evening primrose oil, jojoba oil; fatty alcohols, such as lauryl alcohol, myristyl alcohol, cetyl alcohol, stearyl alcohol, oleyl alcohol, cetyl alcohol; fatty acids, such as myristic acid, stearic acid, palmitic acid, oleic acid, linoleic acid, linolenic acid and saturated, unsaturated and substituted fatty acids different therefrom; waxes, such as beeswax, carnauba wax, candililla wax, spermaceti, and mixtures of the abovementioned oil and fat components.

Suitable cosmetically and pharmaceutically compatible oil and fat components C) are described in Karl-Heinz Schrader, Grundlagen and Rezepturen der Kosmetika [Fundamentals and formulations of cosmetics], 2nd edition, Verlag Huthig, Heidelberg, pp. 319-355, which is hereby incorporated by reference.

The compositions according to the invention can comprise, as active substance, e.g. as cosmetic and/or pharmaceutical active substance B) (and also if appropriate as auxiliary C)), at least one polymer which differs from the ampholytic copolymers A) according to the invention. These include, very generally, anionic, cationic, amphoteric and neutral polymers.

Examples of anionic polymers are homopolymers and copolymers of acrylic acid and methacrylic acid or salts thereof, copolymers of acrylic acid and acrylamide and salts thereof; sodium salts of polyhydroxycarboxylic acids, water-soluble or water-dispersible polyesters, polyurethanes, e.g. Luviset PUR® from BASF, and polyureas. Particularly suitable polymers are copolymers of t-butyl acrylate, ethyl acrylate, methacrylic acid (e.g. Luvimer® 100P), copolymers of ethyl acrylate and methacrylic acid (e.g. Luvimer® MAE), copolymers of N-tert-butylacrylamide, ethyl acrylate, acrylic acid (Ultrahold® 8, strong), copolymers of vinyl acetate, crotonic acid and if appropriate further vinyl esters (e.g. Luviset® grades), maleic anhydride copolymers, if appropriate reacted with alcohol, anionic polysiloxanes, e.g. carboxyfunctional ones, t-butyl acrylate, methacrylic acid (e.g. Luviskol® VBM), copolymers of acrylic acid and methacrylic acid with hydrophobic monomers, such as, for example, C₄-C₃₀-alkyl esters of (meth)acrylic acid, C₄-C₃₀-alkylvinyl esters, C₄-C₃₀-alkyl vinyl ethers and hyaluronic acid. Examples of anionic polymers are also vinyl acetate/crotonic acid copolymers, as are sold, for example, under the names Resyn® (National Starch) and Gafset® (GAF), and vinylpyrrolidone/vinyl acrylate copolymers obtainable, for example, under the trade name Luviflex® (BASF). Further suitable polymers are the vinylpyrrolidone/acrylate terpolymer obtainable under the name Luviflex® VBM-35 (BASF) and polyamides containing sodium sulfonate or polyesters containing sodium sulfonate. Also suitable are vinylpyrrolidone/ethyl methacrylate/methacrylic acid copolymers, as are sold by Stepan under the names Stepanhold-Extra and —R1, and the Carboset® grades from BF Goodrich.

Suitable cationic polymers are, for example, cationic polymers with the INCl name Polyquaternium, e.g. copolymers of vinylpyrrolidone/N-vinylimidazolium salts (Luviquat® FC, Luviquat® HM, Luviquat® MS, Luviset Clear®, Luviquat Supreme®, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamido copolymers (Polyquaternium-7) and chitosan. Suitable cationic (quaternized) polymers are also Merquat® (polymer based on dimethyldiallylammonium chloride), Gafquat® (quaternary polymers which are formed by reacting polyvinylpyrrolidone with quaternary ammonium compounds), Polymer JR (hydroxyethylcellulose with cationic groups) and cationic polymers based on plants, e.g. guar polymers, such as the Jaguar® grades from Rhodia.

Very particularly suitable polymers are neutral polymers, such as polyvinylpyrrolidones, copolymers of N-vinylpyrrolidone and vinyl acetate and/or vinyl propionate, polysiloxanes, polyvinylcaprolactam and other copolymers with N-vinylpyrrolidone, polyethyleneimines and salts thereof, polyvinylamines and salts thereof, cellulose derivatives, polyaspartic acid salts and derivatives. These include, for example, Luviflex® Swing (partially saponified copolymer of polyvinyl acetate and polyethylene glycol, BASF).

Suitable polymers are also nonionic, water-soluble or water-dispersible polymers or oligomers, such as polyvinylcaprolactam, e.g. Luviskol® Plus (BASF), or polyvinylpyrrolidone and copolymers thereof, in particular with vinyl esters, such as vinyl acetate, e.g. Luviskol® VA 37 (BASF); polyamides, e.g. based on itaconic acid and aliphatic diamines, as are described, for example, in DE-A-43 33 238.

Suitable polymers are also amphoteric or zwitterionic polymers, such as the octylacrylamide/methyl methacrylate/tert-butylaminoethyl methacrylate/2-hydroxypropyl methacrylate copolymers obtainable under the names Amphomer® (National Starch), and zwitterionic polymers, as are disclosed, for example, in the German patent applications DE 39 29 973, DE 21 50 557, DE 28 17 369 and DE 37 08 451. Acrylamidopropyltrimethylammonium chloride/acrylic acid or methacrylic acid copolymers and alkali metal and ammonium salts thereof are preferred zwitterionic polymers. Further suitable zwitterionic polymers are methacroylethylbetaine/meth-acrylate copolymers, which are commercially available under the name Amersette® (AMERCHOL), and copolymers of hydroxyethyl methacrylate, methyl methacrylate, N,N-dimethylaminoethyl methacrylate and acrylic acid (Jordapon®).

Suitable polymers are also nonionic, siloxane-containing, water-soluble or water-dispersible polymers, e.g. polyether siloxanes, such as Tegopren® (Goldschmidt) or Belsil® (Wacker).

As already detailed, the copolymers A) are advantageously suitable for stabilizing water-insoluble (or only slightly water-soluble) active substances and effect substances B) in aqueous phase and therefore allow the preparation of aqueous formulations of such active substances and effect substances. They are also suitable for preparing solid formulations of these active substances and effect substances, which can be converted to an aqueous formulation, e.g. commercial form, administration form or active form. This can also take place only after the solid composition has been applied (e.g. in the digestive tract of an organism, etc.).

Within the scope of the present invention, the “solubility improvement” achieved with the polymers A) used according to the invention is therefore understood in the wide sense. It includes, firstly, the stabilization of heterogeneous systems in which the active substance is present as emulsified and/or dispersed phase (disperse phase) in an aqueous medium as continuous phase. It also includes the stabilization of transitional stages to homogeneous solutions, such as colloidal solutions, etc. ranging to molecularly disperse solutions. It also includes an improvement in solubility in the sense of a solubilization during which the sparingly water-soluble or water-insoluble substances are converted into clear, at most opalescent aqueous solutions. Finally, it also includes the ability to form so-called “solid solutions”.

A low (poor) solubility means within the scope of this invention a solubility of the active substance or effect substance in water of less than 10 g/l, in particular of less than 1 g/l and specifically of less than 0.1 g/l at 25° C. and 1013 mbar.

The aqueous active substance compositions of water-insoluble active substances or effect substances prepared using the copolymers A) comprise, besides an aqueous medium as continuous phase, at least one active substance and/or effect substance B) which is dispersed or solubilized in the continuous phase and which has a solubility in water at 25° C./1013 mbar of less than 10 g/l, in particular of less than 1 g/l and specifically of less than 0.1 g/l, and at least one ampholytic copolymer A).

The active substance is present in the continuous aqueous phase in extremely finely divided form. This can, for example, be attributed to the fact that the active substance forms aggregates in the aqueous phase with the polymers A). These aggregates generally have average particle sizes of less than 1 μm, often of less than 500 nm, in particular of less than 400 nm, specifically of less than 300 nm. Depending on the nature of the polymer and of the active substance or effect substance, and depending on the concentration ratios, the aggregates can also be so small that they are no longer present in the form of detectable discrete particles, but in dissolved form (particle size <10 nm).

The particle sizes given here are weight-average particle sizes, as can be ascertained by dynamic light scattering. Methods for this are known to the person skilled in the art, for example from H. Wiese in D. Distler, Wässrige Polymerdispersionen [Aqueous polymer dispersions], Wiley-VCH 1999, chapter 4.2.1, p. 40ff and literature cited therein, and H. Auweter, D. Horn, J. Colloid Interf. Sci. 105 (1985) 399, D. Lilge, D. Horn, Colloid Polym. Sci. 269 (1991) 704 or H. Wiese, D. Horn, J. Chem. Phys. 94 (1991) 6429.

The copolymers A) can be used advantageously as solubilizers for UV absorbers which are insoluble or sparingly soluble in water.

The term UV absorber comprises, in the context of the present invention, UV-A, UV-B and/or broadband filters.

Advantageous broadband filters, UV-A filter substances or UV-B filter substances are, for example, representatives of the following classes of compounds:

Bisresorcinyltriazine derivatives with the following structure:

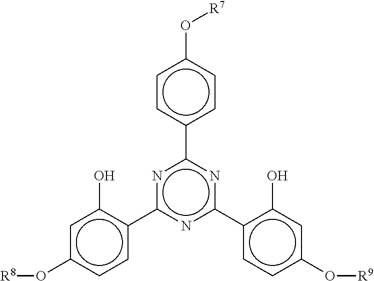

in which R⁷, R⁸ and R⁹ are chosen, independently of one another, from the group of branched and unbranched alkyl groups having 1 to 10 carbon atoms or are a single hydrogen atom. Particular preference is given to 2,4-bis{[4-(2-ethylhexyloxy)-2-hydroxy]phenyl}-6-(4-methoxyphenyl)-1,3,5-triazine (INCl: Aniso Triazine), which can be obtained from CIBA Chemikalien GmbH under the trade name Tinosorb® S.

In addition, other UV filter substances exhibiting the structural unit

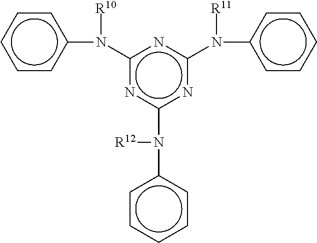

are advantageous UV filter substances for the purposes of the present invention, for example the s-triazine derivatives disclosed in the European Laid-Open Application EP 570 838 A1, the chemical structure of which is represented by the generic formula

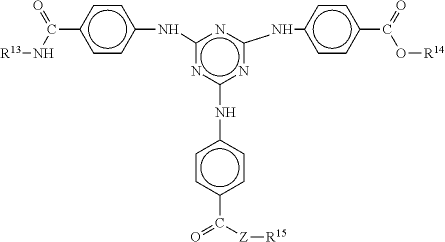

in which
R¹³ is a branched or unbranched C₁-C₁₈-alkyl radical or a C₅-C₁₂-cycloalkyl radical, optionally substituted by one or more C₁-C₄-alkyl groups,
Z is an oxygen atom or an NH group,
R¹⁴ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula

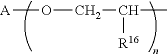

in which
A is a branched or unbranched C₁-C₁₈-alkyl radical, a C₅-C₁₂-cycloalkyl radical or an aryl radical, optionally substituted by one or more C₁-C₄-alkyl groups,
R¹⁶ is a hydrogen atom or a methyl group,
n is a number from 1 to 10,
R¹⁵ is a branched or unbranched C₁-C₁₈-alkyl radical or a C₅-C₁₂-cycloalkyl radical, optionally substituted by one or more C₁-C₄-alkyl groups, if X is the NH group, and is a branched or unbranched C₁-C₁₈-alkyl radical or a C₅-C₁₂-cycloalkyl radical, optionally substituted by one or more C₁-C₄-alkyl groups, or a hydrogen atom, an alkali metal atom, an ammonium group or a group of the formula

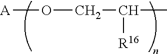

in which
A is a branched or unbranched C₁-C₁₈-alkyl radical, a C₅-C₁₂-cycloalkyl radical or an aryl radical, optionally substituted by one or more C₁-C₄-alkyl groups,
R¹⁶ is a hydrogen atom or a methyl group,
n is a number from 1 to 10,
   if X is an oxygen atom.

Furthermore, a particularly preferred UV filter substance for the purposes of the present invention is an asymmetrically substituted s-triazine, the chemical structure of which is represented by the formula

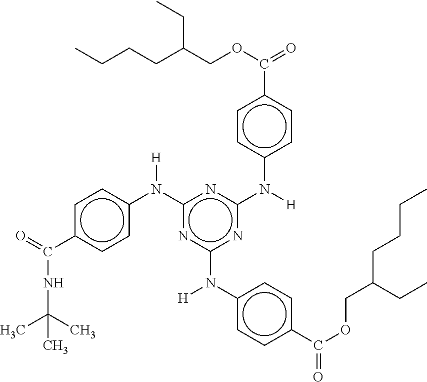

which is also described below as dioctyl butylamido triazone (INCl: Diethylhexylbutamidotriazone) and is available from Sigma 3V under the trade name UVASORB® HEB.

Also advantageous for the purposes of the present invention is a symmetrically substituted s-triazine, 2,4,6-tris[anilino(p-carbo-2′-ethyl-l′-hexyloxy)]-1,3,5-triazine (INCl: Ethylhexyl Triazone), which is sold by BASF Aktiengesellschaft under the trade name UVINUL® T 150.

In addition, European Laid-Open Application 775 698 discloses bisresorcinyltriazine derivatives which are preferably to be used, the chemical structure of which is represented by the generic formula

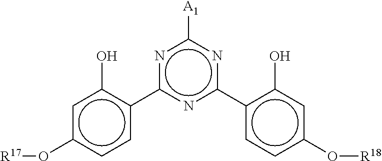

in which R¹⁷ and R¹⁸ are, inter alia, C₃-C₁₈-alkyl or C₂-C₁₈-alkenyl and A₁ is an aromatic radical.

The following compounds are also advantageous for the purposes of the present invention: 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxypropyloxy)-2-hydroxy]phenyl}-6-(4-methoxyphenyl)-1,3,5-triazine, 2,4-bis{[4-(2-ethylhexyloxy)-2-hydroxy]phenyl}-6-[4-(2-methoxyethylcarboxyl)-phenylamino]-1,3,5-triazine, 2,4-bis{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis{[4-tris(trimethylsiloxysilylpropyloxy)-2-hydroxy]phenyl}-6-(4-methoxyphenyl)-1,3,5-triazine, 2,4-bis{[4-(2″-methylpropenyloxy)-2-hydroxy]phenyl}-6-(4-methoxyphenyl)-1,3,5-triazine and 2,4-bis{[4-(1′,1′,1′,3′,5′,5′,5′-heptamethylsiloxy-2″-methylpropyloxy)-2-hydroxy]phenyl}-6-(4-methoxyphenyl)-1,3,5-triazine.

Advantageous oil-soluble UV-B and/or broadband filter substances are, e.g.:
3-benzylidenecamphor derivatives, preferably 3-(4-methylbenzylidene)camphor or 3-benzylidenecamphor;
4-aminobenzoic acid derivatives, preferably
4-(dimethylamino)benzoic acid (2-ethylhexyl) ester or
4-(dimethylamino)benzoic acid amyl ester;
benzophenone derivatives, preferably 2-hydroxy-4-methoxybenzophenone (available from BASF under the trade name Uvinul® M40),
2-hydroxy-4-methoxy-4′-methylbenzophenone, 2,2′-dihydroxy-4-methoxybenzophenone or 2,2′,4,4′-tetrahydroxybenzophenone (available from BASF under the trade name Uvinul® D 50).

Particularly advantageous UV filter substances for the purposes of the present invention which are liquid at ambient temperature are homomethyl salicylate,

2-ethylhexyl 2-cyano-3,3-diphenylacrylate, 2-ethylhexyl 2-hydroxybenzoate and esters of cinnamic acid, preferably 4-methoxycinnamic acid (2-ethylhexyl) ester and 4-methoxycinnamic acid isopentyl ester.

Homomethyl salicylate (INCl: Homosalate) is characterized by the following structure:

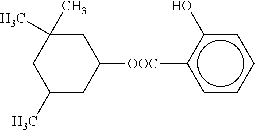

2-Ethylhexyl 2-cyano-3,3-diphenylacrylate (INCl: Octocrylene) is available from BASF under the name Uvinul® N 539T and is characterized by the following structure:

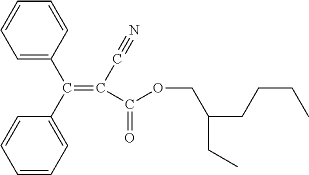

2-Ethylhexyl 2-hydroxybenzoate (2-ethylhexyl salicylate, octyl salicylate, INCl: Ethylhexyl Salicylate) is available, for example, from Haarmann & Reimer under the trade name Neo Heliopan® OS and is characterized by the following structure:

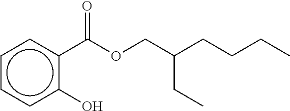

4-Methoxycinnamic acid (2-ethylhexyl) ester (2-ethylhexyl 4-methoxycinnamate, INCl: Ethylhexyl Methoxycinnamate) is, for example, available from BASF under the trade name Uvinul® MC 80 and is characterized by the following structure:

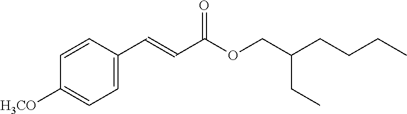

4-Methoxycinnamic acid isopentyl ester (isopentyl 4-methoxycinnamate, INCl: Isoamyl p-Methoxycinnamate) is, for example, available from Haarmann & Reimer under the trade name Neo Heliopan® E 1000 and is characterized by the following structure:

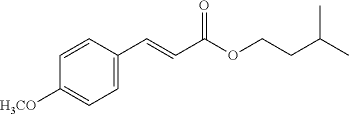

Advantageous dibenzoylmethane derivatives for the purposes of the present invention are, in particular, 4-(tert-butyl)-4′-methoxydibenzoylmethane (CAS No. 70356-09-1), which is sold by BASF under the trade name Uvinul® BMBM and by Merck under the trade name Eusolex® 9020 and which is characterized by the following structure:

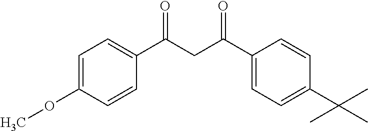

A further advantageous dibenzoylmethane derivative is 4-isopropyl-dibenzoylmethane (CAS No. 63250-25-9), which is sold by Merck under the name Eusolex® 8020. Eusolex 8020 is characterized by the following structure:

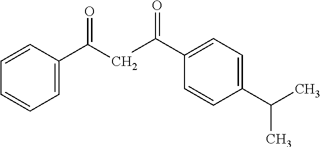

Benzotriazoles are characterized by the following structural formula:

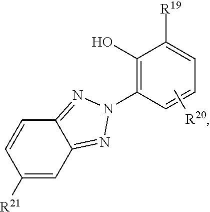

in which

R¹⁹ and R²⁰ are, independently of one another, linear or branched, saturated or unsaturated, substituted (e.g., substituted by a phenyl radical) or unsubstituted alkyl radicals having 1 to 18 carbon atoms.

An advantageous benzotriazole for the purposes of the present invention is furthermore 2-(2H-benzotriazol-2-yl)-4-methyl-6-[2-methyl-3-[1,3,3,3-tetramethyl-1-[(trimethyl-silyl)oxy]disiloxanyl]propyl]phenol (CAS No.: 155633-54-8) with the INCl name Drometrizole Trisiloxane, which is sold by Chimex under the trade name Mexoryl® XL and is characterized by the following structural chemical formula

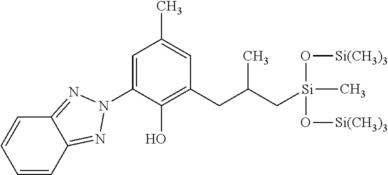

Further advantageous benzotriazoles for the purposes of the present invention are [2,4′-dihydroxy-3-(2H-benzotriazol-2-yl)-5-(1,1,3,3-tetramethylbutyl)-2′-(n-octoxy)-5′-benzoyl]diphenylmethane, 2,2′-methylenebis[6-(2H-benzotriazol-2-yl)-4-(methyl)phenol], 2,2′-methylenebis[6-(2H-benzotriazol-2-yl)-4-(1,1,3,3-tetramethylbutyl)phenol], 2-(2′-hydroxy-5′-octylphenyl)benzotriazole, 2-(2′-hydroxy-3′,5′-di(t-amyl)phenyl)benzo-triazole and 2-(2′-hydroxy-5′-methylphenyl)benzotriazole.

A further UV filter advantageous for the purposes of the present invention is the diphenylbutadiene compound disclosed in EP-A-0 916 335 of the following formula.

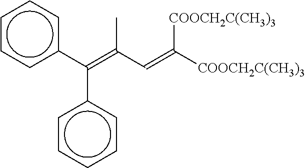

A further UV-A filter advantageous for the purposes of the present invention is the 2-(4-ethoxyanilinomethylene)propanedicarboxylic acid diethyl ester disclosed in EP-A-0 895 776 of the following formula.

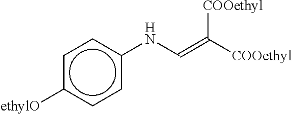

Likewise advantageous for the purposes of the present invention is an amino-substituted hydroxybenzophenone of the following formula:

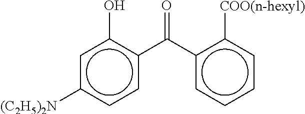

which is sold by BASF Aktiengesellschaft as UV-A filter under the trade name UVINUL® A Plus.

The copolymers A) to be used according to the invention are likewise suitable for the use for modifying the rheological properties, and as solubilizer in pharmaceutical preparations of every type.

The invention therefore further provides a pharmaceutical composition comprising
A) at least one nitrogen atom-containing hyperbranched polymer, as defined above,
B) at least one pharmaceutically acceptable active substance and
C) if appropriate at least one further pharmaceutically acceptable active substance or auxiliary, different from B).

In a specific embodiment, the pharmaceutical compositions comprise at least one pharmaceutically acceptable active substance B) which has a solubility in water at 25° C. and 1013 mbar of less than 10 g/l. For this, the copolymers A) serve as solubilizers for the sparingly soluble active substance(s). The formulation base of the pharmaceutical compositions according to the invention preferably comprises pharmaceutically acceptable auxiliaries. Pharmaceutically acceptable auxiliaries are auxiliaries which are known for use in the field of pharmaceuticals, food technology and related fields, in particular those listed in the relevant pharmacopeias (e.g., DAB, Ph. Eur., BP, NF), and other auxiliaries, the properties of which do not preclude a physiological application.

Suitable auxiliaries can be: lubricants, wetting agents, emulsifying and suspending agents, preservatives, antioxidants, antiirritatives, chelating agents, emulsion stabilizers, film-forming agents, gel formers, odor-masking agents, resins, hydrocolloids, solvents, solubility promoters, neutralizing agents, permeation accelerators, pigments, quaternary ammonium compounds, refatting and superfatting agents, ointment, cream or oil base substances, silicone derivatives, stabilizers, sterilants, propellants, drying agents, opacifiers, thickeners, waxes, softeners or white oils. One embodiment relating to this is based on expert knowledge, as described, for example, in Fiedler, H. P., Lexikon der Hilfsstoffe für Pharmazie, Kosmetik and angrenzende Gebiete [Encyclopedia of Auxiliaries for Pharmaceuticals, Cosmetics and Related Fields], 4th edition, Aulendorf: ECV-Editio-Kantor-Verlag, 1996.

In order to prepare pharmaceutical compositions according to the invention, the active substances can be mixed or diluted with a suitable auxiliary (excipient). Excipients can be solid, semiliquid or liquid materials which can act as vehicle, carrier or medium for the active substance. The admixing of additional auxiliaries is carried out, if desired, in a way known to a person skilled in the art. It relates in this connection in particular to aqueous solutions or solubilizates for oral or parenteral application. In addition, the copolymers to be used according to the invention are also suitable for use in oral administration forms, such as tablets, capsules, powders or solutions. In this connection, they can make the sparingly soluble pharmaceutical available with increased bioavailability. In the parenteral application, emulsions, for example fatty emulsions, can also be used in addition to solubilizates. The copolymers A) according to the invention are also suitable for this purpose, in order to process a sparingly soluble pharmaceutical.

Pharmaceutical formulations of the abovementioned kind can be obtained by processing the copolymers A) to be used according to the invention with pharmaceutical active substances using conventional methods and with the use of known and new active substances.

The use according to the invention can additionally comprise pharmaceutical auxiliaries and/or diluents. Cosolvents, stabilizers and preservatives are especially mentioned as auxiliaries.

The pharmaceutical active substances used are substances which are soluble or insoluble or slightly soluble in water. According to DAB 9 (German Pharmacopeia), the solubility of pharmaceutical active substances is categorized as follows: slightly soluble (soluble in 30 to 100 parts of solvent); sparingly soluble (soluble in 100 to 1000 parts of solvent); virtually insoluble (soluble in more than 10 000 parts of solvent). The active substances can in this connection come from any range indicated.

Particular preference is given to those of the abovementioned pharmaceutical compositions relating to formulations which can be applied parenterally.

The content of copolymer A) in the pharmaceutical compositions is, depending on the active substance, in the range from 0.01 to 50% by weight, preferably 0.1 to 40% by weight, particularly preferably 1 to 30% by weight, based on the total weight of the composition.

In principle, all pharmaceutical active substances and prodrugs are suitable for the preparation of the pharmaceutical compositions according to the invention. These include benzodiazepines, antihypertensives, vitamins, cytostatics, in particular taxol, anesthetics, neuroleptics, antidepressants, antibiotics, antimycotics, fungicides, chemotherapeutics, urologics, thrombocyte aggregation inhibitors, sulfonamides, spasmolytics, hormones, immunoglobulins, sera, thyroid therapeutic agents, psychopharmacological agents, antiparkinsonians and other antihyperkinetic agents, ophthalmics, neuropathy preparations, calcium metabolism regulators, muscle relaxants, narcotics, antilipemics, hepatic therapeutic agents, coronary agents, cardiacs, immunotherapeutics, regulatory peptides and their inhibitors, hypnotics, sedatives, gynecological agents, antigouts, fibrinolytic agents, enzyme preparations and transport proteins, enzyme inhibitors, emetics, circulation-promoting agents, diuretics, diagnostics, corticoids, cholinergics, bile duct therapeutics, antiasthmatics, broncholytics, beta-receptor blockers, calcium antagonists, ACE inhibitors, antiarteriosclerotics, antiinflammatories, anticoagulants, antihypotensives, antihypoglycemics, antihypertonics, antifibrinolytics, antiepileptics, antiemetics, antidotes, antidiabetics, antiarrhythmics, antianemics, antiallergics, anthelmintics, analgesics, analeptics, aldosterone antagonists and slimming agents. Examples of suitable pharmaceutical active substances are in particular the active substances mentioned in paragraphs 0105 to 0131 of US 2003/0157170.

An additional aspect of the present invention relates to the use of the ampholytic copolymers A) as solubilizers in molecularly disperse systems. Solid dispersions, that is homogeneous extremely finely disperse phases of two or more solids, and their special case of “solid solutions” (molecularly disperse systems), and their use in pharmaceutical technology, are generally known (cf. Chiou and Riegelmann, J. Pharm. Sci., 1971, 60, 1281-1300). In addition, the present invention also relates to solid solutions comprising at least one copolymer A) to be used according to the invention.

The preparation of solid solutions can be carried out with the help of melting processes or according to the solution process.

The copolymers according to the invention are suitable as polymeric auxiliary, i.e. solubilizer for the preparation of such solid dispersions or solid solutions.

According to the melting process, for example, an active substance B) and the copolymer A) can be weighed out and mixed in the desired ratio, e.g., in equal parts. A tumbler mixer, for example, is suitable for the mixing. The mixture can subsequently be extruded, e.g. in a twin-screw extruder. The diameter of the cooled product strand thus obtained, consisting of a solid solution of the chosen active substance in the chosen copolymer to be used according to the invention, is dependent on the diameter of the perforation of the perforated plates of the extruder. Cylindrical particles can be obtained by cutting the cooled product strands using a rotating knife, the length of the particles depending on the distance between the perforated plate and the knife. The mean diameter of the cylindrical particles is as a rule approximately 1000 to approximately 3000 μm and the length is as a rule approximately 2000 to approximately 5000 μm. Larger extrudates can be comminuted in a downstream step.

Alternatively, a solid solution can also be prepared in the solution process. For this, the active substance B) and the copolymer A) are usually dissolved in a suitable solvent. Subsequently, the solution is usually poured into a suitable mold and the solvent is removed, for example by drying. The drying conditions are advantageously chosen according to the properties of the active substance (e.g., thermal lability) and solvent (e.g., boiling point).

Taking into consideration the characteristics of the material, the molded article produced or the extrudate, for example, can be comminuted with a suitable mill (e.g., pin mill). The solid solution is advantageously comminuted down to a mean particle size of less than approximately 2000 μm, preferably less than approximately 1000 μm and particularly preferably less than approximately 500 μm.

The bulk material produced can now be processed, with suitable auxiliaries, to give a tableting mixture or to give a capsule feedstock. The tableting is advantageously carried out so that tablets with a hardness of greater than approximately 35 N, preferably greater than approximately 60 N, particularly preferably approximately 80 to approximately 100 N, are obtained.

Like conventional formulations, the formulations thus obtained can, if necessary, be coated with suitable coating materials in order to achieve resistance to gastric juices, delayed release, masking of taste, and the like.

Besides the use in pharmacy, the copolymers A) to be used according to the invention are also suitable in the food sector for modifying the rheological properties and/or as solubilizers for sparingly water-soluble or water-insoluble nutrients, auxiliaries or additives, such as, e.g., fat-soluble vitamins or carotenoids. Mention may be made, as examples, of clear drinks colored with carotenoids. The present invention consequently also provides food preparations comprising at least one of the copolymers A) to be used according to the invention. In the context of the present invention, the food preparations are also to be understood as including food supplements, such as, e.g., preparations comprising food dyes, and dietary foods. Moreover, the specified copolymers A) are also suitable for modifying the rheological properties and/or as solubilizers for feed supplements for animal food.

Furthermore, the ampholytic copolymers A) are suitable for the preparation of aqueous preparations of food supplements such as water-insoluble vitamins and provitamins, such as vitamin A, vitamin A acetate, vitamin D, vitamin E, tocopherol derivatives, such as tocopherol acetate, and vitamin K.

Examples of effect substances which can be formulated as aqueous active substance composition according to the invention are dyes: e.g., the dyes disclosed in DE-A 102 45 209 and the compounds described, according to the Colour Index, as disperse dyes and as solvent dyes, which are also described as dispersion dyes. A list of suitable dispersion dyes is given, for example, in Ullmann's Encyclopedia of Industrial Chemistry, 4th edition, Vol. 10, pp. 155-165 (see also Vol. 7, p. 585ff—Anthraquinone Dyes; Vol. 8, p. 244ff—Azo Dyes; Vol. 9, p. 313ff—Quinophthalone Dyes). This literature reference and the compounds mentioned therein are hereby expressly incorporated by reference. Suitable dispersion dyes and solvent dyes according to the invention comprise the most varied categories of dyes with various chromophores, for example anthraquinone dyes, monoazo and disazo dyes, quinophthalone dyes, methine and azamethine dyes, naphthalimide dyes, naphthoquinone dyes and nitro dyes. Examples of suitable dispersion dyes according to the invention are the dispersion dyes of the following Colour Index list: C. I. Disperse Yellow 1-228, C. I. Disperse Orange 1-148, C. I. Disperse Red 1-349, C. I. Disperse Violet 1-97, C. I. Disperse Blue 1-349, C. I. Disperse Green 1-9, C. I. Disperse Brown 1-21, C. I. Disperse Black 1-36. Examples of suitable solvent dyes according to the invention are the compounds of the following Colour Index list: C. I. Solvent Yellow 2-191, C. I. Solvent Orange 1-113, C. I. Solvent Red 1-248, C. I. Solvent Violet 2-61, C. I. Solvent Blue 2-143, C. I. Solvent Green 1-35, C. I. Solvent Brown 1-63, C. I. Solvent Black 3-50. Suitable dyes according to the invention are furthermore derivatives of naphthalene, of anthracene, of perylene, of terylene or of quarterylene, and diketopyrrolopyrrole dyes, perinone dyes, coumarin dyes, isoindoline and isoindolinone dyes, porphyrin dyes, and phthalocyanine and naphthalocyanine dyes.

In addition to the abovementioned constituents, the active substance and effect substance compositions according to the invention can also comprise conventional surface-active substances and other additives. The surface-active substances include surfactants, dispersing agents and wetting agents. The other additives include in particular thickeners, antifoaming agents, preservatives, antifreeze agents, stabilizers, and the like.

Of use in principle are anionic, cationic, nonionic and amphoteric surfactants, including polymer surfactants and surfactants with heteroatoms in the hydrophobic group.

The anionic surfactants include, for example, carboxylates, in particular alkali metal, alkaline earth metal and ammonium salts of fatty acids, e.g. potassium stearate, which are usually also described as soaps; acyl glutamates; sarcosinates, e.g. sodium lauroyl sarcosinate; taurates; methylcelluloses; alkyl phosphates, in particular mono- and diphosphoric acid alkyl esters; sulfates, in particular alkyl sulfates and alkyl ether sulfates; sulfonates, furthermore alkyl- and alkylarylsulfonates, in particular alkali metal, alkaline earth metal and ammonium salts of arylsulfonic acids and alkyl-substituted arylsulfonic acids, alkylbenzenesulfonic acids, such as, for example, lignin- and phenolsulfonic acid, naphthalene- and dibutylnaphthalenesulfonic acids, or dodecylbenzenesulfonates, alkylnaphthalenesulfonates, alkyl methyl ester sulfonates, condensation products of sulfonated naphthalene and derivatives thereof with formaldehyde, condensation products of naphthalenesulfonic acids, phenol- and/or phenolsulfonic acids with formaldehyde or with formaldehyde and urea, or mono- or dialkylsuccinic acid ester sulfonates; and protein hydrolysates and lignosulfite waste liquors. The abovementioned sulfonic acids are advantageously used in the form of their neutral or, if appropriate, basic salts.

The cationic surfactants include, for example, quaternary ammonium compounds, in particular alkyltrimethylammonium and dialkyldimethylammonium halides and alkyl sulfates, and also pyridine and imidazoline derivatives, in particular alkylpyridinium halides.

The nonionic surfactants include, for example:
fatty alcohol polyoxyethylene esters, for example lauryl alcohol polyoxyethylene ether acetate,
alkyl polyoxyethylene and polyoxypropylene ethers, e.g. of isotridecyl alcohol, and fatty alcohol polyoxyethylene ethers,
alkylaryl alcohol polyoxyethylene ethers, e.g. octylphenol polyoxyethylene ether,
alkoxylated animal and/or plant fats and/or oils, for example corn oil ethoxylates, castor oil ethoxylates or tallow fat ethoxylates,
glycerol esters, such as, for example, glycerol monostearate,
fatty alcohol alkoxylates and oxo alcohol alkoxylates, in particular of the RO—(R₁₈O)ᵣ(R₁₉O)ₛR₂₀ type, with R₁₈ and R₁₉, independently of one another, ═C₂H₄, C₃H₆ or C₄H₈, R₂₀═H or C₁-C₁₂-alkyl, R═C₃-C₃₀-alkyl or C₆-C₃₀-alkenyl, and r and s are, independently of one another, 0 to 50, it not being possible for both to represent 0, such as isotridecyl alcohol and oleyl alcohol polyoxyethylene ether,
alkylphenol alkoxylates, such as, for example, ethoxylated isooctyl-, octyl- or nonylphenol, or tributylphenol polyoxyethylene ether,
fatty amine alkoxylates, fatty acid amide alkoxylates and fatty acid diethanolamide alkoxylates, in particular their ethoxylates,
sugar surfactants, sorbitol esters, such as, for example, sorbitan fatty acid esters (sorbitan monooleate or sorbitan tristearate), polyoxyethylene sorbitan fatty acid esters, alkylpolyglycosides or N-alkylgluconamides,
alkyl methyl sulfoxides,
alkyldimethylphosphine oxides, such as, for example, tetradecyldimethyl-phosphine oxide.

The amphoteric surfactants include, for example, sulfobetaines, carboxybetaines and alkyldimethylamine oxides, e.g. tetradecyldimethylamine oxide.

Further surfactants which should be mentioned here by way of example are perfluorosurfactants, silicone surfactants, phospholipids such as, for example, lecithin or chemically modified lecithins, or amino acid surfactants, e.g. N-lauroyl glutamate.

Unless otherwise specified, the alkyl chains of the abovementioned surfactants are linear or branched radicals having usually 8 to 20 carbon atoms.

In one embodiment, the aqueous active substance compositions according to the invention comprise no more than 10% by weight, preferably no more than 5% by weight and in particular no more than 3% by weight, e.g. 0.01 to 5% by weight or 0.1 to 3% by weight, of conventional surface-active substances, in each case based on the total amount of active substance and polymer composition. The conventional surface-active substances then preferably make up no more than 5% by weight and in particular no more than 3% by weight, e.g. 0.01 to 5% by weight or 0.1 to 3% by weight, based on the total weight of the composition.

However, depending on the use, it may be advantageous for the active substance compositions according to the invention to be formulated with surface-active substances. The proportion of conventional surface-active substance then frequently lies in the range from 0.5 to 30% by weight, in particular in the range from 1 to 20% by weight, based on the total amount of the active substance and polymer composition, or in the range from 0.2 to 20% by weight and in particular in the range from 0.5 to 15% by weight, based on the total weight of the composition formulated.

Even if one advantage of the compositions according to the invention is their low content of volatile organic substances, it may for some applications be desirable to admix the compositions according to the invention with organic solvents, oils and fats, preferably those solvents or oils and fats which are environmentally friendly or biocompatible, e.g. the abovementioned water-miscible solvents or solvents, oils or fats which are immiscible with water or only miscible with water to a very limited extent, e.g.:
paraffin oils, aromatic hydrocarbons and aromatic hydrocarbon mixtures, e.g. xylenes, Solvesso 100, 150 or 200, and the like,
phenols and alkylphenols, e.g. phenol, hydroquinone, nonylphenol, and the like,
ketones with more than 4 carbon atoms, such as cyclohexanone, isophorone, isopherone, acetophenone or acetonaphthone,
alcohols with more than 4 carbon atoms, such as acetylated lanolin alcohol, cetyl alcohol, 1-decanol, 1-heptanol, 1-hexanol, isooctadecanol, isopropyl alcohol, oleyl alcohol or benzyl alcohol,
carboxylic acid esters, e.g. adipic acid dialkyl esters, such as bis(2-ethylhexyl) adipate, phthalic acid dialkyl esters, such as bis(2-ethylhexyl) phthalate, acetic acid alkyl esters (also branched alkyl groups), such as ethyl acetate and ethyl acetoacetate, stearates, such as butyl stearate or glycerol monostearate, citrates, such as tributyl acetylcitrate, in addition cetyl octanoate, methyl oleate, methyl p-hydroxybenzoate, methyl tetradecanoate, propyl p-hydroxybenzoate, methyl benzoate, or lactic acid esters, such as isopropyl lactate, butyl lactate and 2-ethylhexyl lactate,
vegetable oils, such as palm oil, rapeseed oil, ricinus oil and derivatives thereof, such as, e.g., oxidized, coconut oil, cod-liver oil, corn oil, soybean oil, linseed oil, olive oil, peanut oil, safflower oil, sesame seed oil, grapefruit oil, basil oil, apricot oil, ginger oil, geranium oil, orange oil, rosemary oil, macadamia oil, onion oil, mandarin oil, pine oil or sunflower oil,
hydrogenated vegetable oils, such as hydrogenated palm oil, hydrogenated rapeseed oil or hydrogenated soybean oil,
animal oils, such as lard oil or fish oils,
dialkylamides of medium- to long-chain fatty acids, e.g. Hallcomides, and
vegetable oil esters, such as rapeseed oil methyl ester.

The copolymers A) can be used together with conventional thickeners.

Suitable thickeners are compounds which bestow a pseudoplastic flow behavior on the formulation, i.e. high viscosity at rest and low viscosity in the agitated state. Mention may be made, in this connection, for example, of polysaccharides or organic layered minerals, such as Xanthan Gum® (Kelzan® from Kelco), Rhodopol® 23 (Rhone-Poulenc) or Veegum® (R.T. Vanderbilt), or Attaclay® (Engelhardt), with Xanthan Gum® preferably being used.

Silicone emulsions (such as, e.g., Silicone® SRE, from Wacker, or Rhodorsil® from Rhodia), long-chain alcohols, fatty acids, fluoroorganic compounds and their mixtures, for example, come into consideration as antifoam agents suitable for dispersions according to the invention.

Bactericides can be added to stabilize the compositions according to the invention against infection by microorganisms. Suitable bactericides are, for example, Proxel® from ICI or Acticide® RS from Thor Chemie and Kathon® MK from Rohm & Haas.

Suitable antifreeze agents are organic polyols, e.g. ethylene glycol, propylene glycol or glycerol. These are generally used in amounts of no more than 10% by weight, based on the total weight of the active substance composition, in order for the desired content of volatile compounds not to be exceeded. In one embodiment of the invention, the proportion therein of the various volatile organic compounds is preferably no more than 1% by weight, in particular no more than 1000 ppm.

If appropriate, the active substance compositions according to the invention can, to regulate the pH, comprise 1 to 5% by weight of buffer, based on the total amount of the formulation prepared, the amount and the type of the buffer used depending on the chemical properties of the active substance or substances. Examples of buffers are alkali metal salts of weak inorganic or organic acids, such as, e.g., phosphoric acid, boric acid, acetic acid, propionic acid, citric acid, fumaric acid, tartaric acid, oxalic acid and succinic acid.

In a particularly preferred embodiment, the copolymers according to the invention are used as a component in a cosmetic composition. As described previously, they can here serve to modify the rheological properties of a cosmetic composition based on an aqueous medium. They can also serve as solubilizers for cosmetic compositions which comprise at least one cosmetically acceptable active substance or effect substance which has a solubility in water at 25° C. and 1013 mbar of less than 10 g/l. Independently of this, the copolymers A) according to the invention also have good film-forming properties and as such can also be used as cosmetic active substance.

The invention further provides a cosmetic or pharmaceutical composition comprising

α) at least one ampholytic copolymer A), as defined above, and

β) at least one cosmetically or pharmaceutically acceptable carrier.

Preferably, the component β) is chosen from
i) water,
ii) water-miscible organic solvents, preferably C₂-C₄-alkanols, in particular ethanol,
iii) oils, fats, waxes,
iv) esters of C₆-C₃₀-monocarboxylic acids with mono-, di- or trihydric alcohols which are different from iii),
v) saturated acyclic and cyclic hydrocarbons,
vi) fatty acids,
vii) fatty alcohols,
viii) propellant gases,

and mixtures thereof.

Suitable hydrophilic and hydrophobic components β) are those specified above.

Specific suitable cosmetically compatible oil and fat components β) are described in Karl-Heinz Schrader, Grundlagen and Rezepturen der Kosmetika [Fundamentals and formulations of cosmetics], 2nd edition, Verlag Hüthig, Heidelberg, pp. 319-355, which is hereby incorporated by reference.

Preferred hydrophilic carriers β) are chosen from water, 1-, 2- or polyhydric alcohols having preferably 1 to 8 carbon atoms, such as ethanol, n-propanol, isopropanol, propylene glycol, glycerol, sorbitol, etc.

The cosmetic compositions according to the invention may be skin cosmetic, hair cosmetic, dermatological, hygiene or pharmaceutical compositions. On account of their film-forming and thickening properties, the above-described copolymers A) are suitable in particular as additives for hair and skin cosmetics. They are specifically suitable for the formulation of gels.

Preferably, the compositions according to the invention are in the form of a gel, foam, spray, ointment, cream, emulsion, suspension, lotion, milk or paste. If desired, liposomes or microspheres can also be used.

The cosmetically active compositions according to the invention can additionally comprise cosmetically and/or dermatologically active substances and effect substances, and auxiliaries. Of suitability in principle are the abovementioned active substances and effect substances B), and auxiliaries C). In a specific embodiment, the cosmetic compositions according to the invention comprise at least one water-insoluble or only sparingly water-soluble active substance or effect substance.

The cosmetic compositions according to the invention preferably comprise at least one copolymer A) as defined above, at least one carrier β) as defined above and at least one constituent different therefrom which is preferably chosen from cosmetically active substances, emulsifiers, surfactants, preservatives, perfume oils, additional thickeners, hair polymers, hair and skin conditioners, graft polymers, water-soluble or dispersible silicone-containing polymers, photoprotective agents, bleaches, gel formers, care agents, tinting agents, tanning agents, dyes, pigments, consistency regulators, humectants, refatting agents, collagen, protein hydrolysates, lipids, antioxidants, antifoams, antistats, emollients and softeners.

In addition to the copolymers A), suitable conventional thickeners in such formulations are crosslinked polyacrylic acids and derivatives thereof, polysaccharides and derivatives thereof, such as xanthan gum, agar agar, alginates or tyloses, cellulose derivatives, e.g. carboxymethylcellulose or hydroxycarboxymethylcellulose, fatty alcohols, monoglycerides and fatty acids, polyvinyl alcohol and polyvinylpyrrolidone.

Suitable cosmetically and/or dermatologically active substances are, for example, skin and hair pigmentation agents, tanning agents, bleaches, keratin-hardening substances, antimicrobial active substances, photofilter active substances, repellent active substances, hyperemic substances, keratolytic and keratoplastic substances, antidandruff active substances, antiphlogistics, keratinizing substances, active substances which act as antioxidants and/or as free-radical scavengers, skin moisturizing or humectant substances, refatting active substances, deodorizing active substances, sebostatic active substances, plant extracts, antierythimatous or antiallergic active substances and mixtures thereof.

Artificially skin-tanning active substances which are suitable for tanning the skin without natural or artificial irradiation with UV rays are, for example, dihydroxyacetone, alloxan and walnut shell extract. Suitable keratin-hardening substances are generally active substances as are also used in antiperspirants, such as, for example, potassium aluminum sulfate, aluminum hydroxychloride, aluminum lactate, etc. Antimicrobial active substances are used in order to destroy microorganisms and/or to inhibit their growth and thus serve both as preservatives and also as deodorizing substance which reduces the formation or the intensity of body odor. These include, for example, customary preservatives known to the person skilled in the art, such as p-hydroxybenzoates, imidazolidinylurea, formaldehyde, sorbic acid, benzoic acid, salicylic acid, etc. Such deodorizing substances are, for example, zinc ricinoleate, triclosan, undecylenic acid alkylolamides, triethyl citrate, chlorhexidine etc. Suitable photofilter active substances are substances which absorb UV rays in the UV-B and/or UV-A region. Suitable UV filters are those specified above. Also suitable are p-aminobenzoic esters, cinnamic esters, benzophenones, camphor derivatives, and pigments which stop UV rays, such as titanium dioxide, talc and zinc oxide. Suitable repellent active substances are compounds which are able to keep or drive certain animals, in particular insects, away from people. These include, for example, 2-ethyl-1,3-hexanediol, N,N-diethyl-m-toluamide etc. Suitable hyperemic substances, which stimulate blood flow in the skin, are, for example, essential oils, such as dwarf-pine, lavender, rosemary, juniper berry, horse chestnut extract, birch leaf extract, hay flower extract, ethyl acetate, camphor, menthol, peppermint oil, rosemary extract, eucalyptus oil, etc. Suitable keratolytic and keratoplastic substances are, for example, salicylic acid, calcium thioglycolate, thioglycolic acid and its salts, sulfur, etc. Suitable antidandruff active substances are, for example, sulfur, sulfur polyethylene glycol sorbitan monooleate, sulfur ricinol polyethoxylate, zinc pyrithione, aluminum pyrithione, etc. Suitable antiphlogistics, which counteract skin irritations, are, for example, allantoin, bisabolol, dragosantol, camomile extract, panthenol, etc.

The cosmetic compositions according to the invention can comprise, as cosmetic and/or pharmaceutical active substance (and also if appropriate as auxiliary), at least one cosmetically or pharmaceutically acceptable polymer which differs from the copolymers A) according to the invention. These include, quite generally, anionic, cationic, amphoteric and neutral polymers. The abovementioned polymers are hereby incorporated in their entirety by reference.

According to a preferred embodiment, the compositions according to the invention are a skin-cleansing composition.

Preferred skin-cleansing compositions are soaps of liquid to gel-like consistency, such as transparent soaps, luxury soaps, deodorant soaps, cream soaps, baby soaps, skin protection soaps, abrasive soaps and syndets, pasty soaps, soft soaps and washing pastes, liquid washing, shower and bath preparations, such as washing lotions, shower baths and shower gels, foam baths, oil baths and scrub preparations, shaving foams, shaving lotions and shaving creams.

According to a further preferred embodiment, the compositions according to the invention are cosmetic compositions for the care and protection of the skin, nail care compositions or preparations for decorative cosmetics.

Suitable skin cosmetic compositions are, for example, face tonics, face masks, deodorants and other cosmetic lotions. Compositions for use in decorative cosmetics comprise, for example, concealing sticks, stage make-up, mascara and eye shadows, lipsticks, kohl pencils, eye liners, blushers, powder and eyebrow pencils.

Furthermore, the copolymers A) can be used in nose strips for pore cleansing, in antiacne compositions, repellents, shaving compositions, hair-removal compositions, intimate care compositions, foot care compositions, and in baby care.

The skin care compositions according to the invention are, in particular, W/O or O/W skin creams, day and night creams, eye creams, face creams, antiwrinkle creams, moisturizing creams, bleach creams, vitamin creams, skin lotions, care lotions and moisturizing lotions.

Skin cosmetic and dermatological compositions based on the above-described copolymers A) exhibit advantageous effects. The polymers can, inter alia, contribute to the moisturization and conditioning of the skin and to the improvement in the feel of the skin. By adding the polymers according to the invention, a considerable improvement in the skin compatibility can be achieved in certain formulations.

Skin cosmetic and dermatological compositions comprise preferably at least one copolymer A) in a fraction of from about 0.001 to 30% by weight, preferably 0.01 to 20% by weight, very particularly preferably 0.1 to 12% by weight, based on the total weight of the composition.

Particularly photoprotective compositions based on the copolymers A) have the property of increasing the residence time of the UV-absorbing ingredients compared to customary auxiliaries such as polyvinylpyrrolidone.

Depending on the field of use, the compositions according to the invention can be applied in a form suitable for skin care, such as, for example, in the form of a cream, foam, gel, stick, mousse, milk, spray (pump spray or propellant-containing spray) or lotion.

Besides the copolymers A) and suitable carriers, the skin cosmetic preparations can also comprise further active substances and auxiliaries customary in skin cosmetics, as described above. These include preferably emulsifiers, preservatives, perfume oils, cosmetic active substances, such as phytantriol, vitamin A, E and C, retinol, bisabolol, panthenol, photoprotective agents, bleaches, tanning agents, collagen, protein hydrolysates, stabilizers, pH regulators, dyes, salts, thickeners, gel formers, consistency regulators, silicones, humectants, refatting agents and further customary additives.

Preferred oil and fat components of the skin cosmetic and dermatological compositions are the abovementioned mineral and synthetic oils, such as, for example, paraffins, silicone oils and aliphatic hydrocarbons having more than 8 carbon atoms, animal and vegetable oils, such as, for example, sunflower oil, coconut oil, avocado oil, olive oil, lanolin, or waxes, fatty acids, fatty acid esters, such as, for example, triglycerides of C₆-C₃₀-fatty acids, wax esters, such as, for example, jojoba oil, fatty alcohols, vaseline, hydrogenated lanolin and acetylated lanolin, and mixtures thereof.

The polymers according to the invention can also be mixed with conventional polymers, as described above, if specific properties are to be set.

To set certain properties, such as, for example, improving the feel to the touch, the spreading behavior, the water resistance and/or the binding of active substances and auxiliaries, such as pigments, the skin cosmetic and dermatological preparations can additionally also comprise conditioning substances based on silicone compounds. Suitable silicone compounds are, for example, polyalkylsiloxanes, polyarylsiloxanes, polyarylalkylsiloxanes, polyethersiloxanes or silicone resins.

The cosmetic or dermatological preparations are prepared in accordance with customary processes known to the person skilled in the art.

Preferably, the cosmetic and dermatological compositions are in the form of emulsions, in particular in the form of water-in-oil (W/O) emulsions or oil-in-water (O/W) emulsions.

It is, however, also possible to choose other types of formulation, for example hydrodispersions, gels, oils, oleogels, multiple emulsions, for example in the form of W/O/W or O/W/O emulsions, anhydrous ointments and ointment bases, etc.

Emulsions are prepared by known methods. Besides at least one copolymer A), the emulsions usually comprise customary constituents, such as fatty alcohols, fatty acid esters and, in particular, fatty acid triglycerides, fatty acids, lanolin and derivatives thereof, natural or synthetic oils or waxes and emulsifiers in the presence of water. The selection of additives specific to the type of emulsion and the preparation of suitable emulsions is described, for example, in Schrader, Grundlagen and Rezepturen der Kosmetika [Fundamentals and formulations of cosmetics], Hüthig Buch Verlag, Heidelberg, 2nd edition, 1989, third part, which is hereby expressly incorporated by reference.

A suitable emulsion, e.g. for a skin cream etc., generally comprises an aqueous phase which is emulsified by means of a suitable emulsifier system in an oil phase or fat phase. To prepare the aqueous phase, a copolymer A) can be used.

Preferred fat components which may be present in the fatty phase of the emulsions are: hydrocarbon oils, such as paraffin oil, purcellin oil, perhydrosqualene and solutions of microcrystalline waxes in these oils; animal or vegetable oils, such as sweet almond oil, avocado oil, calophylum oil, lanolin and derivatives thereof, castor oil, sesame oil, olive oil, jojoba oil, karite oil, hoplostethus oil; mineral oils whose distillation start point under atmospheric pressure is at about 250° C. and whose distillation end point is at 410° C., such as, for example, vaseline oil; esters of saturated or unsaturated fatty acids, such as alkyl myristate, e.g. isopropyl myristate, butyl myristate or cetyl myristate, hexadecyl stearate, ethyl or isopropyl palmitate, octanoic acid or decanoic acid triglycerides and cetyl ricinoleate.

The fat phase can also comprise silicone oils which are soluble in other oils, such as dimethylpolysiloxane, methylphenylpolysiloxane and the silicone glycol copolymer, fatty acids and fatty alcohols.

Besides the copolymers A), waxes can also be used, such as, for example, carnauba wax, candililla wax, beeswax, microcrystalline wax, ozokerite wax and Ca, Mg and Al oleates, myristates, linoleates and stearates.

In addition, an emulsion according to the invention can be in the form of an O/W emulsion. Such an emulsion usually comprises an oil phase, emulsifiers which stabilize the oil phase in the water phase, and an aqueous phase, which is usually in thickened form. Suitable emulsifiers are preferably O/W emulsifiers, such as polyglycerol esters, sorbitan esters or partially esterified glycerides.

According to a further preferred embodiment, the compositions according to the invention are a shower gel, a shampoo formulation or a bath preparation.

Such formulations comprise at least one copolymer A) and usually anionic surfactants as base surfactants and amphoteric and/or nonionic surfactants as cosurfactants. Further suitable active substances and/or auxiliaries are generally chosen from lipids, perfume oils, dyes, organic acids, preservatives and antioxidants, and thickeners/gel formers, skin conditioners and humectants.

These formulations comprise preferably 2 to 50% by weight, preferably 5 to 40% by weight, particularly preferably 8 to 30% by weight of surfactants, based on the total weight of the formulation.

In the washing, shower and bath preparations it is possible to use all of the anionic, neutral, amphoteric or cationic surfactants which are customarily used in body-cleansing compositions.

Suitable anionic surfactants are, for example, alkyl sulfates, alkyl ether sulfates, alkyl-sulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

These include, for example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mols per mole of alcohol. In addition, alkylamine oxides, mono- or dialkylalkanolamides, fatty acid esters of polyethylene glycols, ethoxylated fatty acid amides, alkyl polyglycosides or sorbitan ether esters are suitable.

Furthermore, the washing, shower and bath preparations can comprise customary cationic surfactants, such as, for example, quaternary ammonium compounds, for example cetyltrimethylammonium chloride.

In addition, the shower gel/shampoo formulations can comprise thickeners, such as, for example, sodium chloride, PEG-55, propylene glycol oleate, PEG-120 methylglucose dioleate and others, and also preservatives, further active substances and auxiliaries and water.

According to a further preferred embodiment, the compositions according to the invention are a hair-treatment composition.

Hair-treatment compositions according to the invention comprise preferably at least one copolymer A) in an amount in the range from about 0.1 to 30% by weight, preferably 0.5 to 20% by weight, based on the total weight of the composition.

Preferably, the hair-treatment compositions according to the invention are in the form of a setting foam, hair mousse, hair gel, shampoo, hair spray, hair foam, end fluids, neutralizers for permanent waves or hot oil treatments. Depending on the field of use, the hair cosmetic preparations can be applied in the form of (aerosol) spray, (aerosol) foam, gel, gel spray, cream, lotion or wax. Hair sprays here comprise both aerosol sprays and also pump sprays without propellant gas. Hair foams comprise both aerosol foams and pump foams without propellant gas. Hair sprays and hair foams comprise preferably predominantly or exclusively water-soluble or water-dispersible components. If the compounds used in the hair sprays and hair foams according to the invention are water-dispersible, they can be used in the form of aqueous microdispersions having particle diameters of usually 1 to 350 nm, preferably 1 to 250 nm. The solids contents of these preparations are usually in a range from about 0.5 to 20% by weight. These microdispersions generally require no emulsifiers or surfactants for their stabilization.

In a preferred embodiment, the hair cosmetic formulations according to the invention comprise
a) 0.05 to 20% by weight of at least one copolymer A),
b) 20 to 99.95% by weight of water and/or alcohol,
c) 0 to 50% by weight of at least one propellant gas,
d) 0 to 5% by weight of at least one emulsifier,
e) 0 to 3% by weight of at least one thickener, and
f) up to 25% by weight of further constituents.

Alcohol is understood as meaning all alcohols customary in cosmetics, e.g. ethanol, isopropanol, n-propanol.

Further constituents are understood as meaning the additives customary in cosmetics, for example propellants, antifoams, interface-active compounds, i.e. surfactants, emulsifiers, foam formers and solubilizers. The interface-active compounds used may be anionic, cationic, amphoteric or neutral. Further customary constituents may also be, for example, preservatives, perfume oils, opacifiers, active substances, UV filters, care substances, such as panthenol, collagen, vitamins, protein hydrolysates, alpha- and beta-hydroxycarboxylic acids, stabilizers, pH regulators, dyes, viscosity regulators, gel formers, salts, humectants, refatting agents, complex formers and further customary additives.

Also included here are all styling and conditioner polymers known in cosmetics which can be used in combination with the polymers according to the invention if very specific properties are to be set.

Suitable conventional hair cosmetic polymers are, for example, the above-mentioned cationic, anionic, neutral, nonionic or amphoteric polymers, which are hereby incorporated by reference.

To set certain properties, the preparations can additionally also comprise conditioning substances based on silicone compounds. Suitable silicone compounds are, for example, polyalkylsiloxanes, polyarylsiloxanes, polyarylalkylsiloxanes, polyethersiloxanes, silicone resins or dimethicone copolyols (CTFA) and amino-functional silicone compounds such as amodimethicone (CTFA).

The polymers according to the invention are particularly suitable as setting agents in hair styling preparations, in particular hair foams.

Emulsifiers which may be used are all of the emulsifiers customarily used in hair foams. Suitable emulsifiers may be nonionic, cationic or anionic or amphoteric.

Examples of nonionic emulsifiers (INCl nomenclature) are laureths, e.g. laureth-4; ceteths, e.g. ceteth-1, polyethylene glycol cetyl ether; ceteareths, e.g. ceteareth-25, polyglycol fatty acid glycerides, hydroxylated lecithin, lactyl esters of fatty acids, alkyl polyglycosides.

Examples of cationic emulsifiers are cetyldimethyl-2-hydroxyethylammonium dihydrogenphosphate, cetyltrimonium chloride, cetyltrimonium bromide, cocotrimonium methylsulfate, quaternium-1 to x (INCl).

Anionic emulsifiers may be chosen, for example, from the group of alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

A preparation suitable according to the invention for styling gels can, for example, have the following composition:
a) 0.1 to 5% by weight of at least one copolymer A),
b) 0 to 5% by weight of at least one cosmetically acceptable water-soluble or water-dispersible hair-setting polymer which is different from A),
c) 80 to 99.85% by weight of water and/or alcohol,
d) 0 to 1% by weight of a gel former different from A),
e) 0 to 20% by weight of further constituents.

Additional gel formers which can be used are all gel formers customary in cosmetics. These include slightly crosslinked polyacrylic acid, for example Carbomer (INCl), cellulose derivatives, e.g. hydroxypropylcellulose, hydroxyethylcellulose, cationically modified celluloses, polysaccharides, e.g. xanthan gum, caprylic/capric triglyceride, sodium acrylate copolymers, polyquaternium-32 (and) Paraffinum Liquidum (INCl), sodium acrylate copolymers (and) Paraffinum Liquidum (and) PPG-1 trideceth-6, acrylamidopropyltrimonium chloride/acrylamide copolymers, steareth-10 allyl ether acrylate copolymers, polyquaternium-37 (and) Paraffinum Liquidum (and) PPG-1 trideceth-6, polyquaternium-37 (and) propylene glycol dicaprate dicaprylate (and) PPG-1 trideceth-6, polyquaternium-7, polyquaternium-44. Crosslinked homopolymers of acrylic acid which are suitable as additional gel formers are commercially available, for example, under the name Carbopol® from BF GOODRICH. Preference is also given to hydrophobically modified crosslinked polyacrylate polymers, such as Carbopol® Ultrez 21 from Noveon. Further examples of anionic polymers which are suitable as gel formers are copolymers of acrylic acid and acrylamide and salts thereof; sodium salts of polyhydroxycarboxylic acids, water-soluble or water-dispersible polyesters, polyurethanes and polyureas. Particularly suitable polymers are copolymers of (meth)acrylic acid and polyether acrylates, where the polyether chain is terminated with a C₈-C₃₀-alkyl radical. These include, for example, acrylate/beheneth-25 methacrylate copolymers, which are available under the name Aculyn® from Rohm and Haas.

The copolymers A) according to the invention can be used in cosmetic preparations as conditioners.

Suitable anionic surfactants for the formulation with the copolymers A) are, for example, alkyl sulfates, alkyl ether sulfates, alkylsulfonates, alkylarylsulfonates, alkyl succinates, alkyl sulfosuccinates, N-alkoyl sarcosinates, acyl taurates, acyl isethionates, alkyl phosphates, alkyl ether phosphates, alkyl ether carboxylates, alpha-olefinsulfonates, in particular the alkali metal and alkaline earth metal salts, e.g. sodium, potassium, magnesium, calcium, and ammonium and triethanolamine salts. The alkyl ether sulfates, alkyl ether phosphates and alkyl ether carboxylates can have between 1 and 10 ethylene oxide or propylene oxide units, preferably 1 to 3 ethylene oxide units, in the molecule.

For example, sodium lauryl sulfate, ammonium lauryl sulfate, sodium lauryl ether sulfate, ammonium lauryl ether sulfate, sodium lauryl sarcosinate, sodium oleyl succinate, ammonium lauryl sulfosuccinate, sodium dodecylbenzenesulfonate, triethanolamine dodecylbenzenesulfonate are suitable.

Suitable amphoteric surfactants are, for example, alkylbetaines, alkylamidopropylbetaines, alkylsulfobetaines, alkyl glycinates, alkyl carboxyglycinates, alkyl amphoacetates or -propionates, alkyl amphodiacetates or -dipropionates.

For example, cocodimethylsulfopropylbetaine, laurylbetaine, cocamidopropylbetaine or sodium cocamphopropionate can be used.

Suitable nonionic surfactants are, for example, the reaction products of aliphatic alcohols or alkylphenols having 6 to 20 carbon atoms in the alkyl chain, which may be linear or branched, with ethylene oxide and/or propylene oxide. The amount of alkylene oxide is about 6 to 60 mols per mole of alcohol. In addition, alkylamine oxides, mono- or dialkylalkanolamides, fatty acid esters of polyethylene glycols, alkyl polyglycosides or sorbitan ether esters are suitable.

Furthermore, the shampoo formulations can comprise customary cationic surfactants, such as, for example, quaternary ammonium compounds, for example cetyltrimethylammonium chloride.

In the shampoo formulations, in order to achieve certain effects, customary conditioners can be used in combination with the copolymers A). These include, for example, the abovementioned cationic polymers with the INCl name Polyquaternium, in particular copolymers of vinylpyrrolidone/N-vinylimidazolium salts (Luviquat® FC, Luviquat® HM, Luviquat® MS, Luviquat® Care), copolymers of N-vinylpyrrolidone/dimethylaminoethyl methacrylate, quaternized with diethyl sulfate (Luviquat® PQ 11), copolymers of N-vinylcaprolactam/N-vinylpyrrolidone/N-vinylimidazolium salts (Luviquat® Hold); cationic cellulose derivatives (Polyquaternium-4 and -10), acrylamide copolymers (Polyquaternium-7). It is also possible to use protein hydrolysates, and conditioning substances based on silicone compounds, for example polyalkylsiloxanes, polyarylsiloxanes, polyarylalkylsiloxanes, polyethersiloxanes or silicone resins. Further suitable silicone compounds are dimethicone copolyols (CTFA) and amino-functional silicone compounds, such as amodimethicone (CTFA). In addition, cationic guar derivatives such as guar hydroxypropyltrimonium chloride (INCl) can be used.

The invention further provides the use of a copolymer A), as defined above, as auxiliary in pharmacy, preferably as or in (a) coating composition(s) for solid medicaments, for modifying rheological properties, as surface-active compound, as or in (an) adhesive(s), and as or in (a) coating composition(s) for the textile, paper, printing and leather industry.

The invention is illustrated in more detail by reference to the following nonlimiting examples.

### EXAMPLES

### General Preparation Procedure A1

### Example 16

### Copolymer of VP/VI-MAA/MAA/MMA/C₁₈-PEG-MA/EGDMA

| | | |
|---|---|---|
| Initial charge: | 412 g | Butyl acetate |
| Feed 1: | 58.5 g | Vinylpyrrolidone |
| | 18.0 g | Vinylimidazole:Methacrylic acid [1:1] |
| | 57.0 g | Methacrylic acid |
| | 15.0 g | Plex-6877 O ® (25% strength C₁₈-PEG-MA |
| | | in methyl methacrylate) |
| | 1.5 g | Ethylene glycol dimethacrylate |
| Feed 2: | 38.2 g | Butyl acetate |
| | 0.15 g | tert-Butyl peroctoate |
| Feed 3: | 95.6 g | Butyl acetate |
| | 0.39 g | tert-Butyl peroctoate |
| Feed 4: | 23 g | Triethanolamine (about 20% based on |
| | | methacrylic acid) |

At 85-88° C., feed 1 and feed 2 were introduced over two hours into a stirred apparatus fitted with reflux condenser, internal thermometer and four separate feed devices. The reaction mixture was then stirred for a further 2 h at about 88° C. Feed 3 was then metered in over 30 minutes and the mixture was after-polymerized for 3 hours at 90° C. After cooling to about 40° C., the product precipitated out as white powder was partially neutralized with triethanolamine (feed 4) over 1 h at 40° C. The powder was filtered off with suction over a suction filter, washed twice with acetone and dried at 40° C. under reduced pressure.

The polymers 1 and 10 to 20 given in table 1 were prepared analogously. Polymer 17 was partially neutralized only after it had been quaternized with triethanolamine.

### General Preparation Procedure A2

### Example 26

### Copolymer of VP/VI-MAA/VI/MMA/C₁₈-PEG-MA/PETAE

| | | |
|---|---|---|
| Initial charge: | 412 g | Butyl acetate |
| Feed 1: | 37.5 g | Vinylpyrrolidone |
| | 9.75 g | Vinylimidazole:methacrylic acid [1:1] |
| | 72.0 g | Vinylimidazole |
| | 30.0 g | Plex-6877 O ® (25% strength C₁₈-PEG-MA |
| | | in methyl methacrylate) |
| | 0.75 g | Pentaerythritol triallyl ether |
| Feed 2: | 38.2 g | Butyl acetate |
| | 0.15 g | tert-Butyl peroctoate |
| Feed 3: | 95.6 g | Butyl acetate |
| | 0.39 g | tert-Butyl peroctoate |
| Feed 4: | 70 g | Methyl chloride |

At 85-88° C., feed 1 and feed 2 were introduced over two hours into a pressurized apparatus fitted with stirrer, reflux condenser, internal thermometer and four separate feed devices. The reaction mixture was stirred for a further 2 h at about 88° C. Feed 3 was then metered in over 30 minutes and the mixture was after-polymerized for 3 hours at 90° C. The product precipitated out as white powder was quaternized with methyl chloride (feed 4) over about 1 h at 90° C. The powder was then filtered off with suction over a suction filter, washed twice with acetone and dried at 40° C. under reduced pressure.

The polymers 2 to 9 and 21 to 50 given in table I were prepared analogously.

The polymers 3, 4, 7 and 22 to 50 given in table 1 below can also be prepared particularly advantageously by the preparation variant B described below. Here, polymers with low residual monomer contents are generally achieved.

TABLE I
VI-MAA/VP precipitation polymers

| | | | | | | | | | | | | | | Neutrali- |
|---|---|---|---|---|---|---|---|---|---|---|---|---|---|---|
| | VP | VCap | VI- | | | | | | C₁₈- | | | | Quaterni- | zation |
| | [% | [% | MAA | MAA | VI | | | MMA^{#} | PEG-MA^{#} | ODVE | | PETAE | zation | with |
| | by | by | [% by | [% by | [% by | DMAEMA | SMA | [% by | [% by | [% by | EGDMA | [% by | with | TEA |
| | wt.] | wt.] | wt.] | wt.] | wt.] | [% by wt.] | [% by wt.] | wt.] | wt.] | wt.] | [% by wt.] | wt.] | CH₃—Cl | DN % |
| 1 | 40 | — | 10 | 49 | — | — | — | — | — | — | 1.0 | — | — | 20 |
| 2 | 43.7 | — | 20 | 35 | — | — | — | — | — | — | 1.3 | — | ≧70% | — |
| 3 | 63.7 | — | 10 | 25 | — | — | — | — | — | — | 1.3 | — | ≧70% | — |
| 4 | 73.7 | — | 10 | 15 | — | — | — | — | — | — | 1.3 | — | ≧70% | — |
| 5 | 33.6 | 10 | 20 | 35 | — | — | — | — | — | — | 1.4 | — | ≧70% | — |
| 6 | 43.8 | 10 | 20 | 25 | — | — | — | — | — | — | 1.2 | — | ≧70% | — |
| 7 | 53.8 | 10 | 15 | 20 | — | — | — | — | — | — | 1.2 | — | ≧70% | — |
| 8 | 33.7 | — | 20 | 35 | — | — | — | 10 | — | — | 1.3 | — | ≧70% | — |
| 9 | 30 | — | 20 | 48.8 | — | — | — | — | — | — | 1.2 | — | ≧70% | — |
| 10 | 30 | — | 15 | 49 | — | — | 5 | — | — | — | 1.0 | — | — | 20 |
| 11 | 35 | — | 10 | 49 | — | — | — | 3.75 | 1.25 | — | 1.0 | — | — | 20 |
| 12 | 40 | — | 5 | 49 | — | — | — | 3.75 | 1.25 | — | 1.0 | — | — | 20 |
| 13 | 39 | — | 6 | 48.8 | — | — | — | 5 | — | — | 1.2 | — | — | 20 |
| 14 | 39 | — | 10 | 40 | — | — | — | 10 | — | — | 1.0 | — | — | 20 |
| 15 | 29 | — | 12 | 38 | — | — | — | 20 | — | — | 1.0 | — | — | 20 |
| 16 | 39 | — | 12 | 38 | — | — | — | 7.5 | 2.5 | — | 1.0 | — | — | 20 |
| 17 | 39 | — | 12 | 40 | — | — | — | — | — | 8 | 1.0 | — | ≧70%*⁾ | 20 |
| 18 | 39 | — | 15 | 30 | — | — | — | 11.25 | 3.75 | — | 1.0 | — | — | 20 |
| 19 | 44 | — | 20 | 33 | — | — | 2 | — | — | — | 1.0 | — | — | 20 |
| 20 | 40 | — | 30 | 26 | — | — | — | — | — | 3 | 1.0 | — | — | 20 |
| 21 | 40 | — | 30 | 26 | — | — | 3 | — | — | — | 1.0 | — | 75% | — |
| 22 | 29.5 | — | 5 | — | 65 | — | — | — | — | — | — | 0.5 | ≧70% | — |
| 23 | 27 | — | 5 | — | 65 | — | 2.5 | — | — | — | — | 0.5 | ≧70% | — |
| 24 | 25 | — | 5 | — | 60 | — | — | 9.5 | — | — | — | 0.5 | ≧70% | — |
| 25 | 25 | — | 4.5 | — | 60 | — | — | 7.5 | 2.5 | — | — | 0.5 | ≧70% | — |
| 26 | 25 | — | 6.5 | — | 48 | — | — | 15 | 5 | — | — | 0.5 | ≧70% | — |
| 27 | 33 | — | 6.5 | — | 40 | — | — | 15 | 5 | — | — | 0.5 | ≧70% | — |
| 28 | 41.5 | — | 8 | — | 30 | — | — | 15 | 5 | — | — | 0.5 | ≧70% | — |
| 29 | 46.5 | — | 8 | — | 25 | — | — | 15 | 5 | — | — | 0.5 | ≧70% | — |
| 30 | 51.5 | — | 8 | — | 20 | — | — | 15 | 5 | — | — | 0.5 | ≧70% | — |
| 31 | 51.5 | — | 8 | — | 20 | — | — | 20 | — | — | — | 0.5 | ≧70% | — |
| 32 | 70 | — | 8 | — | 20 | — | 1.5 | — | — | — | — | 0.5 | ≧70% | — |
| 33 | 65 | — | 10 | — | 20 | — | 4.5 | — | — | — | — | 0.5 | ≧70% | — |
| 34 | 75 | — | 8 | — | 15 | — | — | | — | 1.5 | — | 0.5 | ≧70% | — |
| 35 | 66.5 | — | 8 | — | 15 | — | — | 10 | — | — | — | 0.5 | ≧70% | — |
| 36 | 66.5 | — | 8 | — | 15 | — | — | 7.5 | 2.5 | — | — | 0.5 | ≧70% | — |
| 37 | 56.5 | — | 8 | — | 15 | — | — | 15 | 5 | — | — | 0.5 | ≧70% | — |
| 38 | 71.5 | — | 8 | — | 10 | — | — | 7.5 | 2.5 | — | — | 0.5 | ≧80% | — |
| 40 | 77.5 | — | 6 | — | 6 | — | — | 7.5 | 2.5 | — | — | 0.5 | ≧80% | — |
| 41 | 82.5 | — | 6 | — | 6 | — | — | 3.75 | 1.25 | — | — | 0.5 | ≧80% | — |
| 42 | 85 | — | 5 | — | 4.5 | — | — | 5 | — | — | — | 0.5 | ≧80% | — |
| 43 | 79.5 | — | 10 | — | 10 | — | — | — | — | — | — | 0.5 | ≧70% | — |
| 44 | 69.5 | 10 | 10 | — | 10 | — | — | — | — | — | — | 0.5 | ≧70% | — |
| 45 | 59.5 | 20 | 10 | — | 10 | — | — | — | — | — | — | 0.5 | ≧70% | — |
| 46 | 74.5 | — | 10 | — | 10 | — | — | 3.75 | 1.25 | — | — | 0.5 | ≧70% | — |
| 47 | 74.5 | — | 10 | — | 10 | — | — | — | — | 5 | — | 0.5 | ≧70% | — |
| 48 | 65 | — | 10 | — | 10 | 10 | 4.5 | — | — | — | — | 0.5 | ≧70% | — |
| 49 | 65 | — | 15 | — | — | 10 | — | 9.5 | — | — | — | 0.5 | ≧70% | — |
| 50 | 59.5 | — | 20 | — | — | 10 | — | 7.5 | 2.5 | — | — | 0.5 | ≧70% | — |

VP = N-Vinylpyrrolidone
VCap = N-Vinylcaprolactam
VI-MAA = Monomer composition of vinylimidazole and methacrylic acid
MAA = Methacrylic acid
VI = N-Vinylimidazole
DMAEMA = N,N-Dimethylaminoethyl methacrylate
SMA = Stearyl methacrylate
MMA = Methyl methacrylate
C₁₈-PEG-MA = polyethylene glycol methacrylate terminated with a C₁₈ fatty alcohol
ODVE Octadecyl vinyl ether
EGDMA Ethylene glycol dimethacrylate
PETAE Pentaerythritol triallyl ether
TEA/DN % Triethanolamine/degree of neutralization
*⁾The product was partially neutralized following quaternization with triethanolamine.
^{#}MMA and C₁₈-PEGMA can be used together, e.g. in the form of the commercial product Plex-6877 O ®, Degussa, Germany.

### Example 83

### Variant B: Polymerization of VI/MAA/VP/PETAE Using Two Free-Radical Initiators with Varying Decompositon Temperature

| | | |
|---|---|---|
| Initial charge: | 613 g | Butyl acetate |
| | 1 g | Trigonox ® 101 (2,5-dimethyl-2,5- |
| | | di(tert-butylperoxy)hexane) |
| Feed 1: | 94 g | Vinylpyrrolidone |
| | 102 g | Vinylimidazole |
| | 6.95 g | Methacrylic acid |
| | 1.2 g | Pentaerythritol triallyl ether |
| Feed 2: | 35 g | n-Butyl acetate |
| | 0.2 g | tert-Butyl peroctoate |
| Feed 3 | 175 g | n-Butyl acetate |
| | 1.0 g | tert-Butyl peroctoate |
| Feed 4: | 175 g | n-Butyl acetate |
| | 1.0 g | tert-Butyl peroctoate |

The initial charge was heated to 90° C. under a nitrogen atmosphere in an apparatus fitted with stirrer, reflux counter, internal thermometer and 4 feed devices. Feeds 1 and 2 were added over 3 h and the mixture was stirred at 90° C. for a further 1.5 h. Feed 3 was added at 100° C. in 1 h, and the reaction mixture was stirred for 1 h at this temperature. Feed 4 was then added at 100° C. over the course of 1 h and the mixture was then stirred for a further 2 h at 100° C. The temperature was increased to 125° C. and the mixture was stirred for a further 2 h at this temperature. The resulting white suspension was then quaternized with 50 g of methyl chloride. The product was filtered, washed with acetone and dried under reduced pressure at 70° C.

All products of variant B according to table 2 were prepared in an analogous way.

### Example 73

### Variant C: Polymerization of AA/DMAPMAM/PETAE Using Two Free-Radical Initiators with Varying Decomposition Temperature

| | | |
|---|---|---|
| Initial charge: | 800 g | Ethyl acetate |
| | 1 g | tert-Butyl peroctoate |
| Feed 1: | 125 g | Acrylic acid |
| Feed 2: | 45 g | DMAPMAM |
| | 1.6 g | Pentaerythritol triallyl ether |
| Feed 3 | 80 g | Ethyl acetate |
| | 0.4 g | Lauroyl peroxide |
| Feed 4: | 200 g | Ethyl acetate |
| | 0.4 g | Lauroyl peroxide |

The initial charge was heated to 75° C. under a nitrogen atmosphere and with stirring in an apparatus fitted with stirrer, reflux counter, internal thermometer and 4 feed devices. Feeds 1, 2 and 3 were added over the course of 3 h and the mixture was stirred for a further 2 h at 75° C. Feed 4 was added at 80° C. in 1 h and the mixture was then stirred for a further 1 h. The temperature was increased to 100° C. and the mixture was stirred for a further 3 h at this temperature. The white suspension obtained was then quaternized with 40 g of methyl chloride. The product was filtered, washed with acetone and dried under reduced pressure at 70° C.

All of the products of variant C according to table 2 were prepared analogously.

### Example 52

### Variant D: Polymerization of AA/DMAPMAM/SMA/PETAE Using Two Free-Radical Initiators with Varying Decomposition Temperature

| | | |
|---|---|---|
| Initial charge: | 670 g | Ethyl acetate/cyclohexane (65:35) |
| | 50 g | Feed 1 |
| | 14 g | Feed 2 |
| | 1.5 g | Pentaerythritol triallyl ether |
| | 1.5 g | tert-Butyl peroctoate |
| Feed 1: | 142.5 g | Acrylic acid |
| | 3 g | Stearyl methacrylate |
| | 3 g | Dimethylaminopropylmethacrylamide |
| | 100 g | Ethyl acetate/cyclohexane (65:35) |
| | 4.3 g | Anhydrous K₂CO₃ |
| Feed 2: | 70 g | Ethyl acetate/cyclohexane (65:35) |
| | 0.35 g | Trigonox ® EHP-C75 (75% strength) |
| Feed 3 | 70 g | Ethyl acetate/cyclohexane (65:35) |
| | 1.0 g | Trigonox ® EHP-C75 (75% strength) |

The initial charge was heated to 50° C. under a nitrogen atmosphere and with stirring in an apparatus fitted with stirrer, reflux counter, internal thermometer and three feed devices. Feed 1 was added over the course of 1.5 h and feed 2 was added over the course of 2 h and the mixture was stirred for a further 2 h at 60° C. Feed 3 was added at 60° C. in 1 h and the mixture was then stirred for a further 2 h at 70° C. The temperature was increased to 100° C. and the mixture was stirred for a further 3 h at this temperature. The white suspension obtained was filtered, washed with acetone and dried under reduced pressure at 70° C.

All of the products of variant D according to table 2 were prepared analogously.

TABLE 2

| | | | | | | | | | | | | Preparation |
|---|---|---|---|---|---|---|---|---|---|---|---|---|
| Ex. | VP^{#} | MAA^{#} | AA^{#} | VI^{#} | DMAPMAM^{#} | SMA^{#} | n-BA^{#} | EMA^{#} | PLEX-O^{#} | EGDMA^{#} | PETAE^{#} | variant |
| 51 | — | 90 | — | 4 | — | 5 | — | — | — | 1.0 | — | C |
| 52 | — | — | 95 | — | 2 | 2 | — | — | — | — | 1.0 | D |
| 53 | — | 85 | — | 10 | — | 3.5 | — | — | — | 1.5 | — | C |
| 54 | — | — | 92 | — | 4 | 2.5 | — | — | — | — | 1.5 | D |
| 55 | — | — | 85 | — | 12 | 1.8 | — | — | — | — | 1.2 | C |
| 56 | — | 85 | — | 5 | 5 | 3.8 | — | — | — | 1.2 | — | C |
| 57 | — | 78 | — | 10 | 7 | 3.8 | — | — | — | 1.2 | — | B |
| 58 | — | — | 75 | — | 10 | — | — | 13.5 | — | — | 1.5 | C |
| 59 | — | 75 | — | — | 23.5 | — | — | — | — | 1.5 | — | C |
| 60 | — | 70 | — | — | 23.5 | — | — | — | 5 | 1.5 | — | C |
| 61 | — | 45 | — | — | 48.5 | — | — | — | 5 | 1.5 | — | C |
| 62 | — | 50 | — | 25 | 23.8 | — | — | — | — | 1.2 | — | C |
| 63 | — | 50 | — | 25 | 20 | 3.8 | — | — | — | 1.2 | — | C |
| 64 | — | 45 | — | — | 38.5 | — | 15 | — | — | 1.5 | — | C |
| 65 | — | 45 | — | — | 38.5 | — | 10 | — | 5 | 1.5 | — | C |
| 66 | — | 45 | — | — | 33.5 | — | 15 | — | 5 | 1.5 | — | C |
| 67 | — | 45 | — | — | 33.5 | — | 25 | — | — | 1.5 | — | C |
| 68 | — | 45 | — | — | 28.5 | — | 25 | — | — | 1.5 | — | C |
| 69 | — | 45 | — | — | 28 | — | 25 | — | — | 2 | — | C |
| 70 | — | 45 | — | — | 23.5 | — | 30 | — | — | 1.5 | — | C |
| 71 | — | 40 | — | — | 23.5 | — | 35 | — | — | 1.5 | — | C |
| 72 | — | 70 | — | — | 28.8 | — | — | — | — | 1.2 | — | C |
| 73 | — | — | 70 | — | 28.8 | — | — | — | — | — | 1.2 | C |
| 74 | 25 | 70 | — | — | 3.8 | — | — | — | — | 1.2 | — | B |
| 75 | 25 | — | 70 | — | 3.8 | — | — | — | — | 1.2 | — | B |
| 76 | 25 | 50 | — | — | 23.8 | — | — | — | — | 1.2 | — | C |
| 77 | 25 | — | 50 | — | 23.8 | — | — | — | — | — | 1.2 | C |
| 78 | 45 | 50 | — | — | 3.8 | — | — | — | — | 1.2 | — | C |
| 79 | 45 | — | 50 | — | 3.8 | — | — | — | — | — | 1.2 | C |
| 80 | 45 | 30 | — | — | 23.8 | — | — | — | — | 1.2 | — | C |
| 81 | 45 | — | 30 | — | 23.8 | — | — | — | — | 1.2 | — | C |
| 82 | 61 | 3.3 | — | 35 | — | — | — | — | — | — | 0.7 | B |
| 83 | 46 | 3.3 | — | 50 | — | — | — | — | — | — | 0.7 | B |
| 84 | 31 | 3.3 | — | 65 | — | — | — | — | — | — | 0.7 | B |
| 85 | 16 | 3.3 | — | 80 | — | — | — | — | — | — | 0.7 | B |
| 86 | — | 4.5 | — | 85 | — | — | — | 10 | — | — | 0.5 | B |
| 87 | — | 5.5 | — | 94 | — | — | — | — | — | — | 0.5 | B |
| 88 | — | 5.5 | — | 90 | — | 4 | — | 4 | — | — | 0.5 | B |
| 89 | — | 3.3 | — | 93 | — | 3 | — | 3 | — | — | 0.7 | B |
| 90 | — | 2.3 | — | 95 | — | 2 | — | 2 | — | — | 0.7 | B |

Ex. Example
^{#}The quantitative data are in % by weight, based on the unsaturated compounds used for the polymerization
VP Vinylpyrrolidone
MAA Methacrylic acid
AS Acrylic acid
VI Vinylimidazole
DMAPMAM Dimethylaminopropylmethacrylamide
SMA Stearyl methacrylate
EGDMA Ethylene glycol dimethacrylate
PETAE Pentaerythritol triallyl ether
EMA Ethyl methacrylate
n-BA n-Butyl acrylate
PLEX-O Plex ® 6877-0 = Methacrylic acid ester of a C₁₆-C₁₈-fatty alcohol alkoxylated with 25 mol of ethylene oxide as 25% strength solution in methyl methacrylate

1. An ampholytic copolymer A) obtainable by free-radical copolymerization of
a) at least one compound with a free-radically polymerizable, α,β-ethylenically unsaturated double bond and at least one anionogenic and/or anionic group per molecule,
b) at least one compound which is chosen from N-vinylimidazole compounds, N-[3-(dimethylamino)propyl]acrylamide, N-[3-(dimethylamino)propyl]methacrylamide and mixtures thereof,
c) at least one free-radically polymerizable crosslinking compound which comprises at least two α,β-ethylenically unsaturated double bonds per molecule.

2. The ampholytic copolymer according to claim 1, where at least part of the compounds a) and b) is used in the form of a monomer pair, where the molar ratio of anionogenic groups of component a) to cationogenic groups of component b) is about 1:1.

3. The ampholytic copolymer A) according to claim 1, obtainable by free-radical copolymerization in accordance with the method of precipitation polymerization.

4. The ampholytic copolymer A) according to claim 2, where the amount of compounds used for the polymerization in the monomer pair is at least 1% by weight.

5. The ampholytic copolymer A) according to claim 2, where the component a) or b) used in deficit is used completely as component of the monomer pair.

6. The ampholytic copolymer A) according to claim 1, where, for the copolymerization, monomers with ionogenic or ionic groups are used in amounts such that the copolymer A) has a molar excess of anionogenic/anionic groups compared with cationogenic/cationic groups or a molar excess of cationogenic/cationic groups compared with anionogenic/anionic groups of at least 1.2:1.

7. (canceled)

8. The ampholytic copolymer according to one of claims 1 to 6, where the component b) is at least one N-vinylimidazole compound, where, for the copolymerization, monomers with ionogenic or ionic groups are used in amounts such that the copolymer A) has a molar excess of anionogenic/anionic groups compared with cationogenic/cationic groups or a molar excess of cationogenic/cationic groups compared with anionogenic/anionic groups of more than 2:1.

9. (canceled)

10. The ampholytic copolymer A) according to claim 1, which additionally comprises, in copolymerized form, at least one further monomer d) which is chosen from α,β-ethylenically unsaturated compounds containing amide groups and of the general formula I 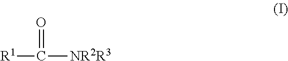
where
one of the radicals R¹ to R³ is a group of the formula CH₂═CR⁴—, where R⁴═H or C₁-C₄-alkyl, and the other radicals R¹ to R³, independently of one another, are H, alkyl, cycloalkyl, heterocycloalkyl, aryl or hetaryl,
where R¹ and R², together with the amide group to which they are bonded, may also be a lactam having 5 to 8 ring atoms,
where R² and R³, together with the nitrogen atom to which they are bonded, may also be a five- to seven-membered heterocycle,
with the proviso that the sum of the carbon atoms of the radicals R¹, R² and R³ is at most 8.

11. The ampholytic copolymer A) according to claim 1, which additionally comprises, in copolymerized form, at least one compound e) which is chosen from compounds of the general formulae III a), III b), III c), III d) and III e) 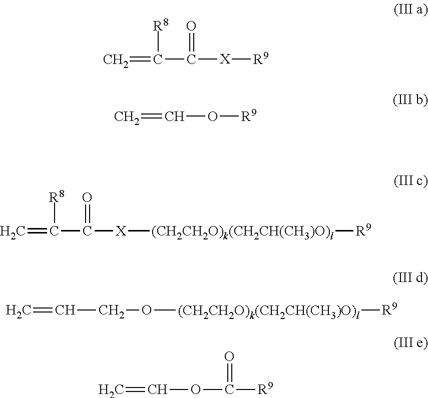
in which
the order of the alkylene oxide units is arbitrary,
k and l, independently of one another, are an integer from 0 to 1000, where the sum of k and l is at least 5,
R⁸ is hydrogen or C₁-C₄-alkyl,
R⁹ is C₈-C₃₀-alkyl or C₈-C₃₀-alkenyl, and
X is O or a group of the formula NR¹⁰, in which R¹⁰ is H, alkyl, alkenyl, cycloalkyl, heterocycloalkyl, aryl or hetaryl.

12. The ampholytic copolymer A) according to claim 1, which in addition to component b) comprises, in copolymerized form, at least one compound f) different from component b) and having a free-radically polymerizable, α,β-ethylenically unsaturated double bond and at least one cationogenic and/or cationic group per molecule.

13. The ampholytic copolymer A) according to claim 1, which additionally comprises, in copolymerized form, at least one compound g) which is chosen from esters of α,β-ethylenically unsaturated mono- and dicarboxylic acids with C₁-C₇-alkanols, esters of α,β-ethylenically unsaturated mono- and dicarboxylic acids with C₂-C₃₀-diols, amides of α,β-ethylenically unsaturated mono- and dicarboxylic acids with C₂-C₃₀-amino alcohols which have a primary or secondary amino group, polyether acrylates different from IIIc), esters of vinyl alcohol and allyl alcohol with C₁-C₇-monocarboxylic acids, C₁-C₇-alkyl vinyl ethers, allyl alcohol alkoxylates different from III d), vinylaromatics, vinyl halides, vinylidene halides, C₂-C₈-monoolefins, nonaromatic hydrocarbons with at least two conjugated double bonds and mixtures thereof.

14. The copolymer A) according to claim 1, which comprises, in copolymerized form,
2 to 96% by weight of at least one compound a) with a free-radically polymerizable α,β-ethylenically unsaturated double bond and at least one anionogenic and/or anionic group per molecule,
2 to 96% by weight of at least one compound b) which is chosen from N-vinylimidazole compounds, N-[3-(dimethylamino)propyl]acrylamide, N-[3-(dimethylamino)propyl]methacrylamide and mixtures thereof,
0.05 to 5% by weight of at least one crosslinker c),
0 to 95% by weight of at least one amide-group-containing monomer d),
0 to 40% by weight of at least one compound e) which is chosen from C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof,
0 to 40% by weight of at least one monomer g) which is chosen from C₁-C₆-(meth)acrylates and mixtures thereof.

15-29. (canceled)

30. The copolymer according to claim 1, obtainable by free-radical copolymerization of
at least 2% by weight, based on the total weight of the monomers used for the polymerization, of at least one monomer pair of N-vinylimidazole and acrylic acid and/or methacrylic acid,
3 to 70% by weight of at least one monomer with a cationogenic or cationic group, chosen from vinylimidazole compounds, N-[3-(dimethylamino)propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker c),
0 to 95% by weight of vinylpyrrolidone and/or vinylcaprolactam d),
0 to 40% by weight of at least one further monomer which is chosen from methyl(meth)acrylate, ethyl(meth)acrylate, n-butyl(meth)acrylate, C₈-C₂₂-(meth)acrylates, C₈-C₂₂-alkyl vinyl ethers, polyether(meth)acrylates terminated with C₈-C₂₂-alkyl groups, allyl alcohol alkoxylates terminated with C₈-C₂₂-alkyl groups, C₈-C₂₂-carboxylic acid vinyl esters and mixtures thereof.

31. (canceled)

32. The copolymer according to claim 1, obtainable by free-radical copolymerization of
at least 5% by weight, based on the total weight of the monomers used for the polymerization, of at least one monomer pair of N-vinylimidazole and acrylic acid and/or methacrylic acid,
3 to 70% by weight of at least one monomer with a cationogenic or cationic group, chosen from vinylimidazole compounds, N-[3-(dimethylamino)propyl]acrylamide, N-[3-(dimethylamino)propyl]methacrylamide, N,N-dimethylaminoethyl(meth)acrylate, N,N-dimethylaminopropyl(meth)acrylate and mixtures thereof,
0.1 to 2% by weight of at least one crosslinker c),
20 to 85% by weight of vinylpyrrolidone and/or vinylcaprolactam d),
1 to 20% by weight of at least one further monomer which is chosen from C₈-C₃₀-(meth)acrylates, polyether(meth)acrylates terminated with C₈-C₃₀-alkyl groups, and mixtures thereof, in particular from stearyl methacrylate, polyethylene glycol(meth)acrylates terminated with C₁₈-C₂₂-alkyl groups, and mixtures thereof.

33-34. (canceled)

35. A process for the preparation of an ampholytic copolymer A) as defined in claim 1 by free-radical copolymerization in accordance with the method of precipitation polymerization.

36. The process according to claim 35, where the polymerization takes place in an anhydrous, aprotic solvent or solvent mixture.

37. (canceled)

38. The process according to claim 35, in which, for the copolymerization, at least two initiators are used whose decomposition temperatures are different from one another by at least 10° C.

39. The process according to claim 38, where the copolymerization takes place until the conclusion of the precipitation of the copolymer at a temperature greater than or equal to the lower decomposition temperature and less than the higher decomposition temperature and, after the precipitation, a further reaction takes place at a temperature greater than or equal to the higher decomposition temperature.

40. The process according to claim 35, comprising a first polymerization phase at a first polymerization temperature and a second polymerization phase at a second polymerization temperature above the first polymerization temperature, where, for the polymerization, at least two initiators are used whose half-lives at the first polymerization temperature differ in such a way that at least one of these initiators decomposes into free radicals during the first polymerization phase and at least one of these initiators essentially does not decompose into free radicals during the first polymerization phase and decomposes into free radicals during the second polymerization phase.

41-55. (canceled)

56. A cosmetic or pharmaceutical composition comprising
A) at least one ampholytic copolymer as defined in claim 1,
B) at least one cosmetically or pharmaceutically acceptable active substance or effect substance, and
C) optionally at least one further cosmetically or pharmaceutically acceptable active substance or auxiliary different from B).
